(19) United States
(12) Patent Application Publication
(10) Pub. No.: US 2006/0142784 A1
(43) Pub. Date: Jun. 29, 2006

(54) Device and method for suturing internal structures puncture wounds

(76) Inventor: Stavros Kontos, Montvale, NJ (US)

Correspondence Address: FITZPATRICK CELLA HARPER & SCINTO, 30 ROCKEFELLER PLAZA, NEW YORK, NY, 10112, US

(21) Appl. No.: 11/092,856

(22) Filed: Mar. 30, 2005

Related U.S. Application Data
(63) Continuation-in-part of application No. 11/022,838, filed on Dec. 28, 2004, now abandoned.

(51) Int. Cl. A61B 17/10

(52) U.S. Cl. 606/139

(57) ABSTRACT

A device for remotely suturing an internal structure of a body and a method of performing said suturing procedure. The device includes one or more anchors and means, operable from outside the body, for forcing said anchors out of said device. The anchors are located in or on said device so that they can be positioned adjacent said internal structure and when forced out of said device, enter and pass through said structure. The anchors are further provided with sutures that can be tightened from outside of the body to secure the anchors in place. In one aspect of the invention, tightening the suture or sutures pulls the anchors toward one another, thereby closing a wound in said internal structure. In another aspect of the invention, tightening of the sutures secures a prosthetic device in place.

This is a continuation-in-part application of U.S. application Ser. No. 11/022,838 filed Dec. 28, 2004. The present invention is directed generally to devices and methods for suturing internal structures, for example, closing and sealing puncture wounds made to access veins or arteries during percutaneous procedures or for purposes of otherwise repairing internal structures or attaching prostheses thereto.

### BACKGROUND OF THE INVENTION

In recent years, an increasing number of surgical and other medical procedures have been performed using minimally invasive techniques. Some minimally invasive techniques, for example, diagnostic and interventional cardiac catheterization procedures, involve gaining access to a remote region of a vein or artery or to an internal organ percutaneously, i.e., through the femoral artery or vein, usually by inserting a catheter or other instrument though the skin and into the artery or vein by puncturing the blood vessel wall. Obviously, when such a procedure is completed, it is necessary to seal the puncture wound to prevent bleeding and promote healing.

When such percutaneous procedures were initially performed, the puncture wounds were traditionally sealed by applying direct pressure to the wound site by a physician or other trained professional until bleeding stopped. Especially when the procedure required insertion of a relatively large instrument, such as the introducer sheath employed when performing intraortic balloon pumping, to stop the bleeding simply by the application of direct pressure often took a considerable amount of time, was frequently uncomfortable for the patient and involved considerable risk of complications such as thrombosis. In addition, it was not unusual, using that method, for bleeding to resume after pressure was removed, thereby necessitating reapplication of pressure and the attendant problems mentioned above.

More recently, methods and devices have been developed to avoid the need for the application of direct pressure. One such method involves insertion of a plug of biocompatible material, such as collagen, into the wound track leading to the puncture sight so as to cover the puncture wound in the blood vessel. This method proved to be less than satisfactory, in part, because it was difficult to locate the plugs properly and, even when properly located, they had a tendency to move and, as a result, not form a good seal.

Another system that was developed to try and physically cover the puncture is shown in U.S. Pat. No. 5,676,689 (“the '689 patent”). The system shown in the '689 patent employs an anchor that is inserted into the lumen of the artery and is held tight against the inside wall of the artery by a suture that passes out of the artery lumen and into the wound track through the tissue overlying the artery. The anchor is intended to be seated in the puncture wound on the inside of the artery wall. In addition, the '689 patent employs a collagen plug that slides on the suture and is pushed against the outside of the artery wall over the puncture sight. The artery wall is, thus, sandwiched between the anchor on the inside of the lumen and the collagen plug which is held against the outside of the artery wall.

A further improvement over the anchor/collagen plug system of the '689 patent is shown in U.S. Pat. No. 6,428,549 (“the '549 patent”) issued to the present inventor. The '549 patent describes a novel method and device for sealing the puncture wound in the artery by suturing the artery wall closed, rather than leaving the puncture open and trying to cover it with a plug.

It is understood that a company called Angiolink Corporation of Taunton, Mass. has recently developed a device, sold under the trademark EVS, for closing puncture wounds using staples.

Other minimally invasive procedures involve gaining access to an internal organ of the body by insertion of trocars and the like during laproscopic procedures. Generally, suturing internal structures during such procedures may be time consuming, involve the use of two or more instruments and the success thereof is often largely dependent on the skill of the physician.

### SUMMARY OF THE INVENTION

One aspect of the present invention represents an improvement over the invention shown in the '549 patent in that the present invention, like the invention of the '549 patent does not rely on trying to cover the open puncture wound but, rather, provides a device and method for suturing the puncture wound closed. The present invention accomplishes this objective by use of device that is simpler and more elegant than the device of the '549 patent and is more easily used, requiring less manipulation by the surgeon.

The device and the method of the present invention are susceptible to many applications. For example, they can be used to close punctures in blood vessels following a percutaneous procedure. In another aspect, the invention can be used to close openings in internal organs, for example atrial septal defects. It can also be used to repair aortic aneurisms by suturing grafts over the affected region. Yet another use would be to attach prostheses, such as heart valves, annuloplasty rings and valvuloplasty rings in place.

The present invention comprises a device for remotely suturing an internal structure of a body comprising an insertion device, one or more anchors slidably held in recesses in said device, each of said anchor recesses being associated with an anchor ejecting opening, suture means connected to said anchors, said suture means being sufficiently long so as to reach from said internal structure to the outside of said body, and means for ejecting said anchors from said recesses through said anchor ejection openings, said anchor ejecting means being operable from outside said body, said anchors being adapted to puncture the wall of said internal structure, a slide mechanism on said suture means to permit securing said anchors in place against said internal structure when the operator pulls said suture tight.

An embodiment of the present invention which is designed to seal punctures in an internal structure of a body comprises an insertion device, two or more anchors slidably held in recesses in said device, a suture connecting said anchors and means for ejecting said anchors from said recesses, said anchors being adapted to puncture said internal structure, a slide mechanism on said suture to permit pulling said anchors toward one another when tension is applied to one end of said suture thereby closing said puncture.

More particularly, this embodiment of the present invention is directed to a device for remotely sealing a puncture in an anatomical structure comprising a catheter or other tubular member (hereinafter referred to simply as a “catheter”) having recesses therein in which reside two or more anchors that can be pushed out of the recesses, by means of push or pull rods (sometimes referred to herein as movement rods), through the anatomical structure on opposite sides of the puncture. The anchors are preferably mounted on a common suture. The two ends of the suture are preferably brought together in a slip knot, with one end passing through the slip knot and out of the patient's body to a point where the physician can grab it and, at the appropriate time, tighten the suture by pulling on the exposed end. Both the anchors and the suture are preferably made of of resorbable material, as is well known in the art.

Once the anchors are forced out of the insertion tube and through the anatomical structure, in one preferred embodiment, barbs on the anchors prevent them from passing back through the structure. The device is then withdrawn, leaving the anchors inside the body, adjacent the anatomical structure on opposite sides of the wound therein. They are held tight against the anatomical structure by the suture and when the slip knot is tightened, the wound in the anatomical structure is closed. Thereafter, the opening seals itself and both the anchors and the suture are subsequently resorbed, leaving no foreign objects in the body.

Another aspect of the present invention is directed to a method for remotely suturing an internal anatomical structure in a body, from outside said body, comprising the steps of:
a. from outside of said body, inserting into said body a device comprising at least one anchor, at least one suture, at least one anchor recess and at least one anchor ejection opening,
b. positioning said device so that at least one of said anchor ejection openings is adjacent said structure,
c. Puncturing a hole in said anatomical structure;
d. causing an anchor to exit at least one of said anchor ejection openings and pass through on of said puncture holes in said anatomical structure, each of said anchors being mounted on a suture,
e. withdrawing said device from said body, leaving at least one of said anchors mounted on said at least one suture adjacent said structure, and
f. from outside of the said body, tightening said at least one suture to secure said anchors in place.

More particularly, one method of the present invention involves closing an opening in anatomical structure comprising the steps of inserting into the opening an insertion device, forcing out of a distal portion of said device two or more anchors which are caused to pass through the wall of the anatomical structure adjacent said opening, pulling the anchors tight against the wall of the structure by use of a suture that is attached to both anchors, removing the insertion device and tightening up on a slide means on the suture to secure the anchors in place and draw the sides of the opening together until the opening seals itself.

In another aspect of the present invention, each anchor is mounted on its own suture and supported on the sutures is a prosthetic device, like an annuloplasty ring, a heart valve or a mesh material. Each suture is tightened independently of the others, although they may be tightened substantially simultaneously, and when tightened and secure in place, so too is the prosthesis. In this embodiment, it is preferred that the anchors and sutures not be made of resorbably material.

### BRIEF DESCRIPTION OF THE DRAWINGS

FIG. 1 is a side view, in cross section, of one embodiment of the insertion device of the instant invention.

FIG. 2 shows a top view of one embodiment of the anchor of the instant invention.

FIG. 2A shows an end view of the proximal end of the anchor of FIG. 2.

FIG. 3 shows a side view of the anchor of FIG. 2.

FIG. 4 shows another embodiment of the anchor of the instant invention.

FIG. 4A shows an end view of the proximal end of the anchor of FIG. 4.

FIG. 5 shows yet another embodiment of the anchor of the instant invention.

FIG. 6 shows a fourth embodiment of the anchor of the instant invention.

FIG. 7 shows one embodiment of two anchors of the instant invention mounted on a suture prior to the anchors being inserted into their respective recesses in the insertion device.

FIG. 7A shows an alternative embodiment of the anchors mounted on the suture.

FIG. 7B shows yet another embodiment of an anchor mounted on a suture.

FIG. 8 shows a partially cross-sectioned view of a blood vessel within a body of a patient with an indwelling guidewire therein.

FIG. 9 shows a partially cross sectioned view of a blood vessel in the body with a device according to one embodiment of the instant invention inserted into the blood vessel of FIG. 8 after the guidewire has been removed and the anchors are in the process of being inserted through the wall of the blood vessel.

FIG. 10 shows the blood vessel of FIG. 8 after the insertion device of the instant invention has been removed, leaving the anchors inside the blood vessel.

FIG. 11 shows the blood vessel of FIG. 8 with the suture, according to one embodiment of the instant invention, having been tightened to pull the anchors and the sides of the opening together.

FIG. 12 shows an alternative embodiment of the instant invention wherein a pusher is used to tighten the slip knot thereby to hold the anchors against the inside wall of the vessel and to pull the sides of the opening together.

FIG. 13 shows an alternative embodiment of the interface between the pushrod and the anchor.

FIG. 14 shows yet another alternative embodiment of the interface between the pushrod and the anchor.

FIG. 15 is a perspective, partially sectioned, of another embodiment of the insertion device of the instant invention.

FIG. 16 is a cross-sectioned side view of the embodiment of FIG. 15.

FIG. 17 is a view of the embodiment if FIG. 15 taken along line A-A of FIG. 16.

FIG. 18 shows the embodiment of FIG. 15, partially sectioned, inserted into a blood vessel with one anchor partially ejected from its recess and in the process of passing through the wall of the blood vessel.

FIG. 19 shows the embodiment of FIG. 15, partially sectioned, inserted into a blood vessel, with the first anchor inside the blood vessel and the second anchor in the process of being inserted through the wall of the blood vessel.

FIG. 20 shows the blood vessel puncture having been closed by the suture inserted using the device of FIG. 15.

FIG. 21 is a cross sectioned side view of an insertion device of another embodiment of the instant invention designed to suture a movable or unstable internal structure.

FIG. 22 is a view of the device of FIG. 21 taken along line B-B of FIG. 21.

FIG. 23 is a side view of an embodiment of the present invention that is intended for affixing a prosthetic device or the like to an internal structure of a body.

FIG. 24 is a cross sectioned view of the device of FIG. 23 taken along line D-D.

FIG. 25 is a cross sectioned view of the device of FIG. 23 taken along line C-C.

FIG. 26 is a cross sectioned view of the device of FIG. 23 taken along line E-E.

FIG. 27 shows the left side of a heart with a valvuloplasty ring being sutured to the mitral valve annulus between the left atrium and the left ventricle.

FIG. 28 shows the left side of a heart with a valvuloplasty ring having been sutured to the mitral valve annulus between the left atrium and the left ventricle.

FIG. 29 is a cross sectioned side view of another embodiment of the present invention intended for closing a hole in an internal structure of the body.

FIG. 30 shows the embodiment of FIG. 29 in place and ready to have the anchors ejected and pushed through the wall of the internal structure.

FIG. 31 shows an alternative embodiment of the device of the present invention.

FIG. 32 shows, in cross section, a view of the embodiment of FIG. 31 taken along line G-G.

FIG. 33 shows, in cross section, a view of the embodiment of FIG. 31 taken along line F-F.

FIG. 34 shows, in cross section, a view of the embodiment of FIG. 31 taken along line H-H.

FIG. 35 shows, in cross section, a view of the embodiment of FIG. 31 Similar to that of FIG. 34, but with the anchors mounted on their own sutures and showing a prosthesis.

FIG. 36 shows, in cross section, a view of the embodiment of FIG. 35taken along line K-K.

FIG. 37 shows, in cross section, a view of the embodiment of FIG. 35 taken along line J-J.

FIG. 38 shows, in cross section, a view of the embodiment of FIG. 35 taken along line H-H.

FIG. 39 shows a valvuloplasty ring with sutures according to the present invention passing through holes in the annulus of the ring.

FIG. 40 is a side view, in cross section, of another embodiment of the insertion device of the instant invention designed to have the anchors outside the structure being sutured.

FIG. 41 shows a partially cross sectioned view of a blood vessel in the body with a device according to the embodiment of FIG. 40 inserted into the blood vessel of FIG. 8 after the guidewire has been removed and the anchors are in the process of being inserted through the wall of the blood vessel.

FIG. 42 shows the blood vessel of FIG. 8 after the insertion device of the embodiment of FIG. 40 has been removed, leaving the anchors outside the blood vessel.

FIG. 43 shows the blood vessel of FIG. 8 after the suture of the embodiment of FIG. 40 has been pulled tight so that the wound is closed and the anchors are outside the blood vessel wall.

FIG. 44 is a side view, in cross section, of another embodiment of the insertion device of the instant invention similar to the embodiment of FIG. 40 except that in the FIG. 44 embodiment, needles are used to puncture the wall of the internal structure.

FIG. 45 is a side view, in cross section, of another embodiment of the insertion device of the invention similar to the embodiment of FIG. 40 except that the in the FIG. 45 embodiment a protruding barb is used as the locating means instead of the indicator lumen of the FIG. 40 embodiment.

FIG. 46 shows a partially cross sectioned view of a blood vessel in the body with a device according to the embodiment of FIG. 45 inserted into the blood vessel with the locating barb extended and splayed outwardly from the body of the insertion tube and the anchors are in the process of being inserted through the wall of the blood vessel.

### DETAILED DESCRIPTION OF THE INVENTION

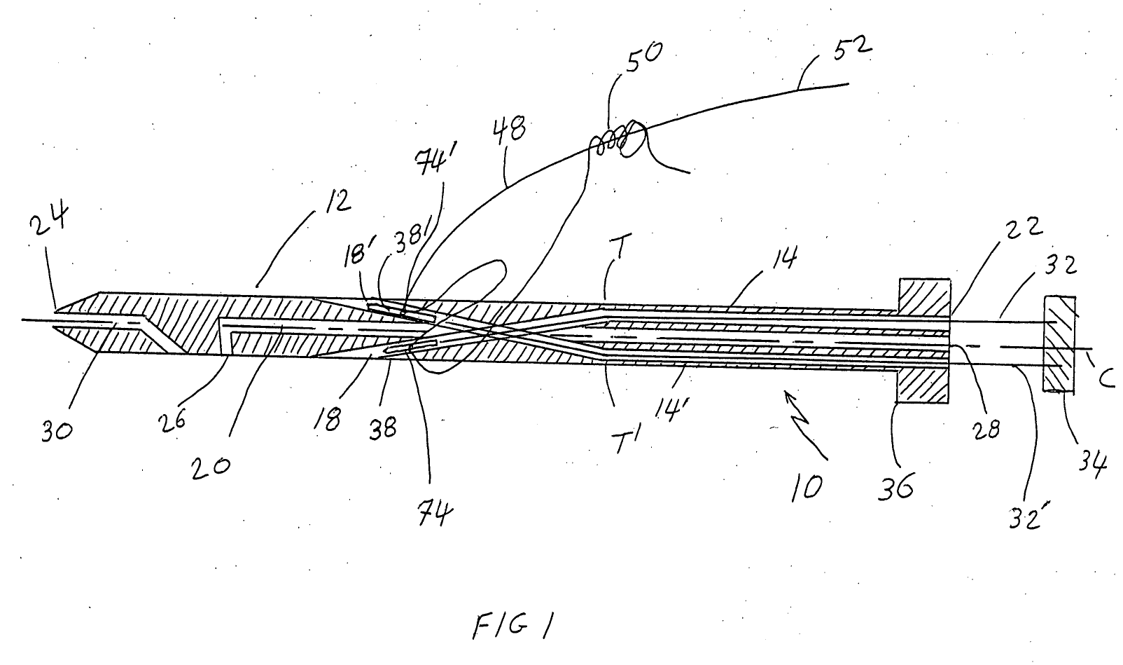

Referring to the drawings, wherein like numbers identify like parts, FIG. 1 shows a device 10 according to one embodiment of the instant invention designed for closing punctures and other openings in blood vessels and other internal organs. This embodiment of the invention will be described with particular reference to sealing a puncture wound in an artery or vein. It should be understood, however, that the instant invention can be employed to close punctures, tears and other holes in many other internal structures and organs, as well as to repair other types of wounds and defects, such as aneurisms, in internal organs by suturing them. As used herein, including in the claims, the term “wound” should be understood to refer to any puncture, tear, hole or other wound or defect in an internal organ or other structure that it is desired to close or otherwise repair or correct or to which it is desired to attach a prosthesis.

Device 10 comprises an elongated, preferably flexible, catheter 12 having a distal end 24 and a proximal end 22, an imaginary center line C and two pushrod lumens 14 and 14′, one located on each side of centerline C. As used herein, “proximal” refers to the end closest to the physician or other operator and “distal” refers to the end toward the patient. Each pushrod lumen begins at the proximal end 22 of catheter 12, runs generally parallel to center line C and then makes a turn at T and T′ to form an oblique angle, and then crosses center line C. Distal ends of lumens 14 and 14′ serve as anchor recesses 74 and 74′ respectively. Anchor recesses 74 and 74′ terminate at their distal ends in openings 18 and 18′ respectively on opposite sides of catheter 12. Preferably, the pushrod lumens are preferably of constant diameter throughout. However, that is not a necessary attribute of the invention. For example, they may be provided with shoulders (not shown) to define the proximal ends of the anchor recesses and to prevent the anchors from being inserted farther than necessary into the lumens. Alternatively, the anchor recesses can have diameters that are larger or smaller than the diameters of the remainder of the pushrod lumens.

Primarily for ease of manufacture, the pushrod lumens are preferably round but, once again, that is not a necessary attribute of the invention. Those lumens may be of any convenient cross sectional shape, including oval, square, octagonal or any other. The only requirement is that the lumens be of such size and shape as to permit the pushrods to slide easily therein.

The pushrods 16 and 16′ are preferably made of nitinol and stainless steel, but any material that will permit them to perform their intended function can be used.

Catheter 12 also contains a flashback or indicator lumen 20 that exits out the side of catheter 12 at opening 26, distally of openings 18 and 18′. Lumen 20 begins at opening 28 in the proximal end of catheter 12 and passes through the catheter to communicate with opening 26, so as to permit fluid communication between distal opening 26 and proximal opening 28. A flexible transparent or translucent plastic tube (not shown) can be attached to the proximal end of catheter 12 to communicate with lumen 20. The cross-sectional shape of the indicator lumen is not important so long as it permits fluid to flow from its distal end to its proximal end. Finally, catheter 12 is also provided with a guidewire lumen 30.

It should be understood that, although the preferred embodiment comprises an indicator lumen, such is not required in order to practice the invention. For example, insertion device 10 can be located properly by use of fluoroscopy or other visualizing technique. Alternatively, a measurement could be taken of the distance from the skin puncture to the point of repair and a mark made on the outside of catheter 20 to let the physician know when the device is in place.

Slidably engaged in lumens 14 and 14′ are pushrods 32 and 32′ respectively. The pushrods may of the same shape as the pushrod lumens, but that is not necessary. For example, the pushrods may be round while the pushrod lumens may be square or octagonal. The only requirement is that the rods be able to slide within the lumens. Preferably, pushrods 32 and 32′ are both attached at their proximal ends to an operating handle 34 so that both pushrods can be operated simultaneously. However, it is certainly within the scope of the instant invention to permit the pushrods to be operated independently, as is shown, for example, in the embodiment of FIG. 15 described below. A protruding shoulder 36 is also provided at the proximal end of catheter 12. Pushrods 32 and 32′ are of sufficient length to reach from proximal end 22 of catheter 12 to openings 18 and 18′ respectively when handle 34 is seated against shoulder 36.

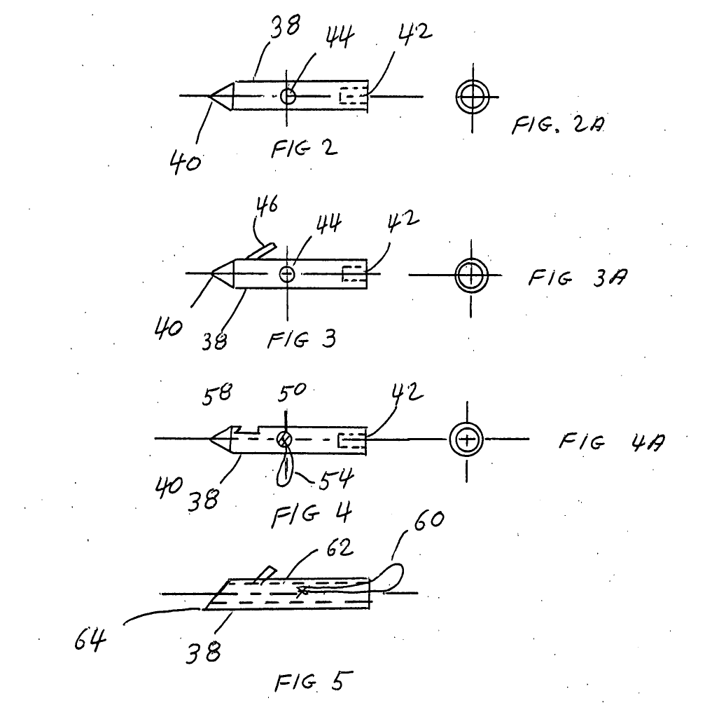

When ready for use, device 10 has two anchors, 38 and 38′ resident in the anchor recess 74 and 74′. For ease of understanding, the description provided below will refer only to anchor 38, but it should be understood that the same description applies to anchor 38′. As best seen in FIGS. 2-5, anchor 38 is a relatively short cylindrical element that has a point 40 at its distal end and a receiving recess 42 at its proximal end. As can be seen from FIG. 2A, anchor 38 is preferably round with recess 42 also being round. It will be apparent to those of skill in the art that the outside surfaces of the anchors can be made to have any shape, so long as they fit and can slide within the anchor recesses 74 and 74′. Similarly, receiving recess 42 can have any shape so long as it can accommodate the distal end of pushrod 14. Although in the preferred embodiment the anchors are provided with receiving recesses to accommodate the distal ends of the pushrods, such receiving recesses are not a necessary attribute of the instant invention. Instead, for example, the proximal end of the anchor can have a flat face that simply abuts the distal tip of the pushrod when the latter is pushed forward. Alternatively, the pushrods can be provided with receiving recesses (as shown in FIG. 13) to accommodate the proximal ends of the anchors.

Anchors 38 and 38′ may be long enough so that when resident in their recesses, their proximal ends extend proximally beyond turns T and T′. In that case, at least the proximal portions of anchors 38 and 38′ would have to be made of a relatively flexible material to enable them to negotiate turns T and T′.

Preferably, anchor 38 is also provided with a barb 46 that lies generally flat against the side of the anchor when the anchor is in anchor recess 74,. In addition, anchor 38 is provided with a transverse hole 44 therethrough. As is best seen in FIG. 7, anchors 38 and 38′ are prepared for insertion into anchor recesses 74 and 74′ by first being threaded onto a suture 48. The ends of suture 48 are brought together and a slip knot 50 is tied in one end, while the other end, 52, is passed through slip knot 50 to permit bringing the anchors toward one another by pulling on end 52.

FIG. 7A shows an alternative embodiment of anchors 38 and 38′ mounted on a common suture 48. In this embodiment, instead of a slip knot, the ends of suture 48 are passed through crimp ring or ferrule 196, with both ends 52 and 52′ passing through and being slidable thereon. Tightening would then be accomplished by pulling on both ends 52 and 52′. As will be readily apparent to those of skill in this art, one of the ends, for example end 52′, could be affixed to ring 196, in which event the suture would be tightened simply by pulling on end 52. Alternatively, both anchors can be mounted on a common suture, but each anchor can be secured to the suture as by use of a knot or glue or some other mechanical attachment, thereby preventing the anchors from sliding on the suture.

FIG. 7B shows yet another embodiment of an anchor being attached to a suture. In this embodiment, each anchor is provided with its own suture, rather than both being mounted on a common suture. In this embodiment, suture 48 is affixed to anchor 38 at 198, for example, by use of a knot or glue or other mechanical fastening, and both ends of the suture pass through crimp ferrule 196 which is slidable thereon, as shown by double headed arrow X. FIG. 7B also shows a prosthetic device, 162 mounted on suture 48 intermediate between crimp ferrule 196 and anchor 38, as will be described below.

Yet another alternative embodiment (not shown) of an anchor being mounted on the suture could combine embodiments 7 and 7A or 7B. This embodiment would employ a slide device, like a simple ring or wafer with holes in it, through which the suture passes, coupled with a slip knot proximal to the slide device.

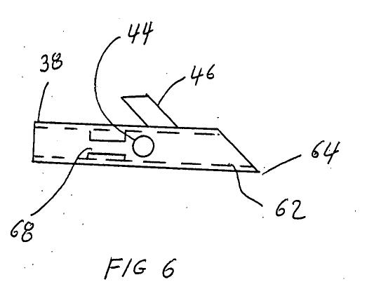

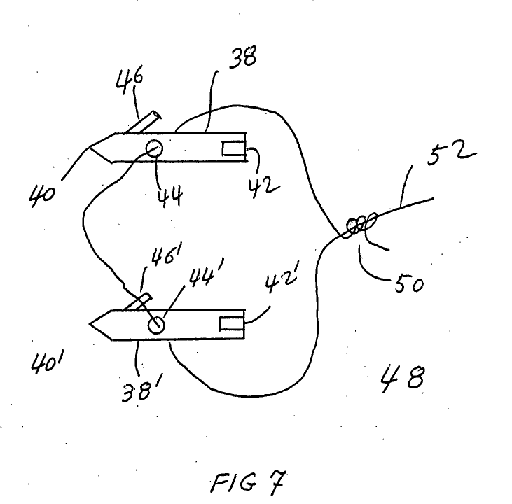

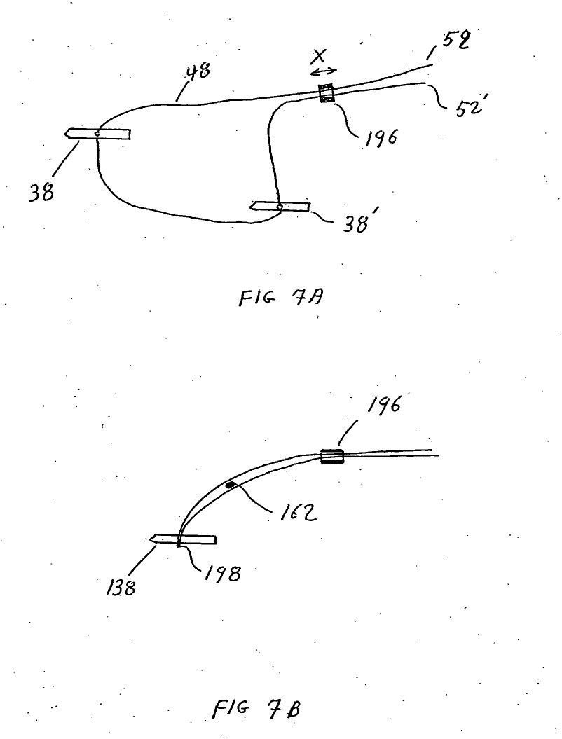

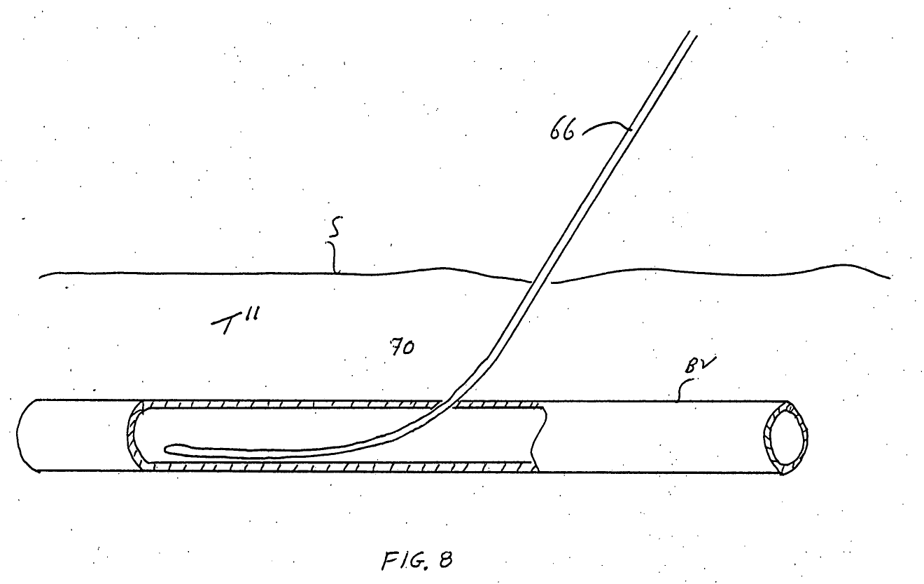

Alternative embodiments of anchor 38 are shown in FIGS. 4, 4A, 5 and 6. In the embodiment of FIGS. 4 and 4A, transverse through hole 44 has been replaced by filament loop 54 attached at a mid-point along the body of anchor 38, and protruding barb 46 has been replaced by recess 56 having an internal barb 58 at the distal end thereof. In the embodiment of FIG. 5, transverse through hole 44 has been replaced by filament loop 60 attached at its distal end inside lumen 62 that passes through the anchor, said filament 60 exiting lumen 62 through the proximal end of anchor 38. The distal end of the anchor of the FIG. 5 embodiment is cut at an acute angle to present a sharp tip 64. The embodiment of FIG. 6 is similar to the embodiment of FIG. 5 except that in the FIG. 6 embodiment an internal shoulder 68 is provided in lumen 62 and instead of filament loop 60, the embodiment of FIG. 6 is provided with transverse hole 44 similar to the transverse hole shown in FIG. 2.

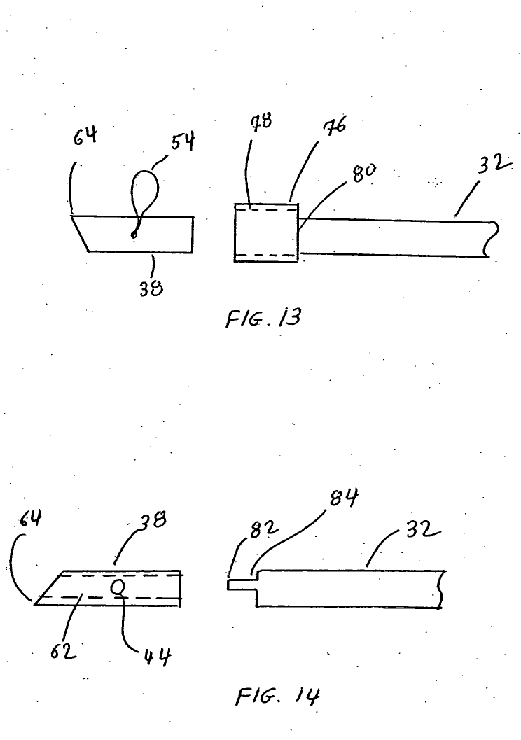

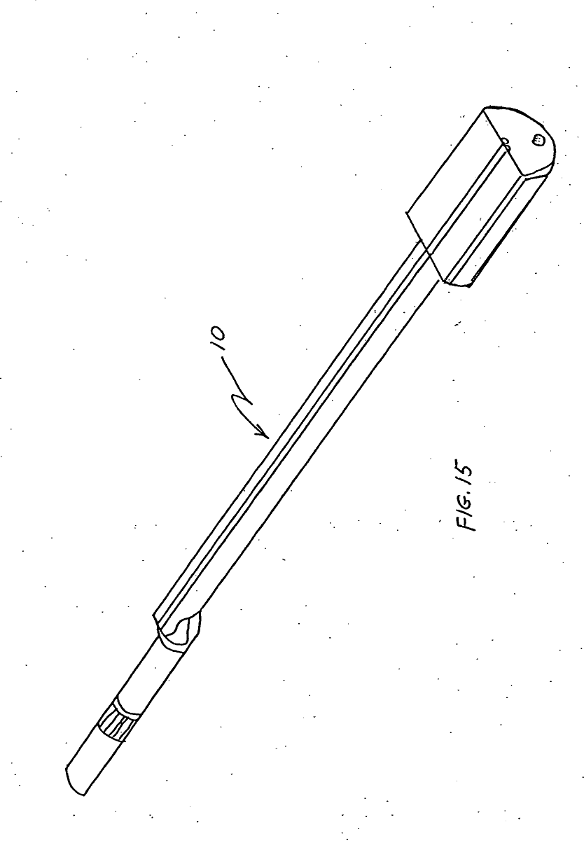

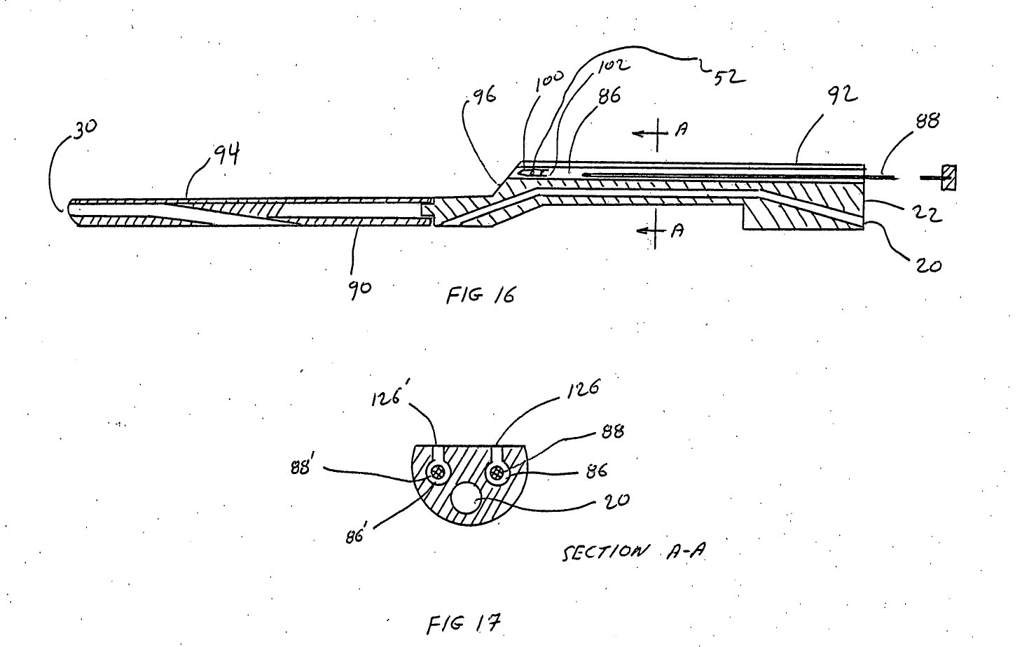

Alternative embodiments of the interface between the pushrod and the anchor are shown in FIGS. 13 and 14. In FIG. 13 pushrod 32 is shown with its distal end 76 enlarged. Within that enlarged distal end is recess 78 which is sized to accommodate therein the proximal end of the anchor 38. When the pushrod is urged forward, the proximal end of anchor 38 enters recess 78 until the proximal end of the anchor abuts the proximal end 80 of recess 78. Thereafter, force applied to the pushrod is transmitted to the anchor. In FIG. 14, pushrod 38 is shown with a reduced diameter distal end 82 which is sized so as to permit it to be accommodated within lumen 62 in anchor 38. In this embodiment, as the pushrod is urged forward, tip 82 enters lumen 62 until shoulder 84 on the pushrod abuts the proximal end of the anchor. Thereafter, force applied to the push rod is transmitted to the anchor.

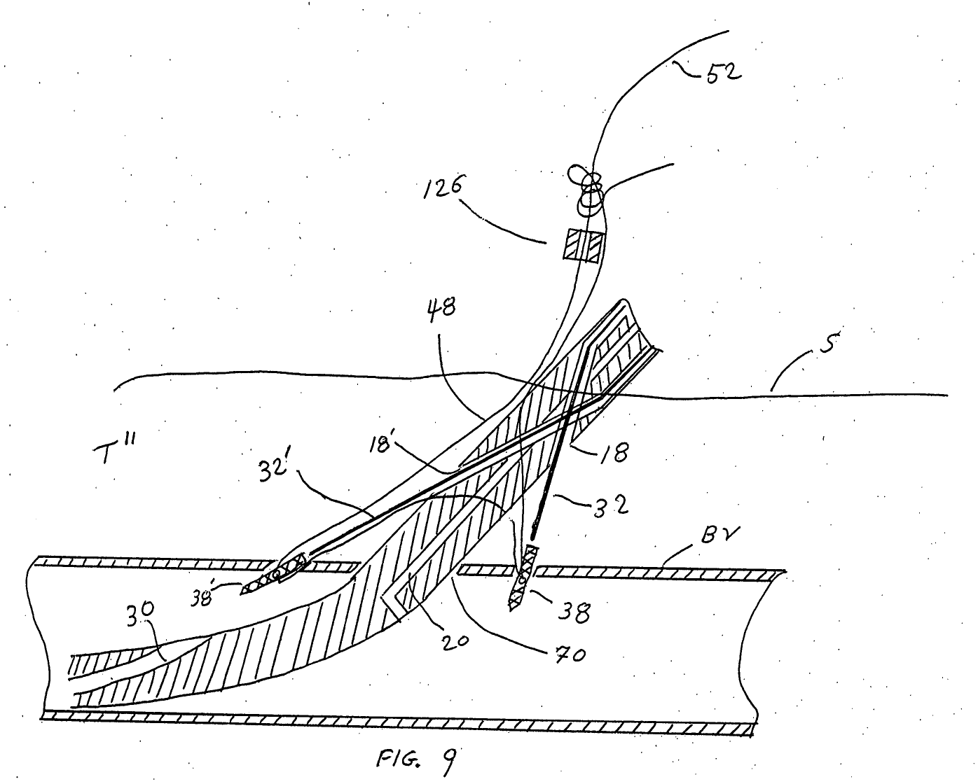

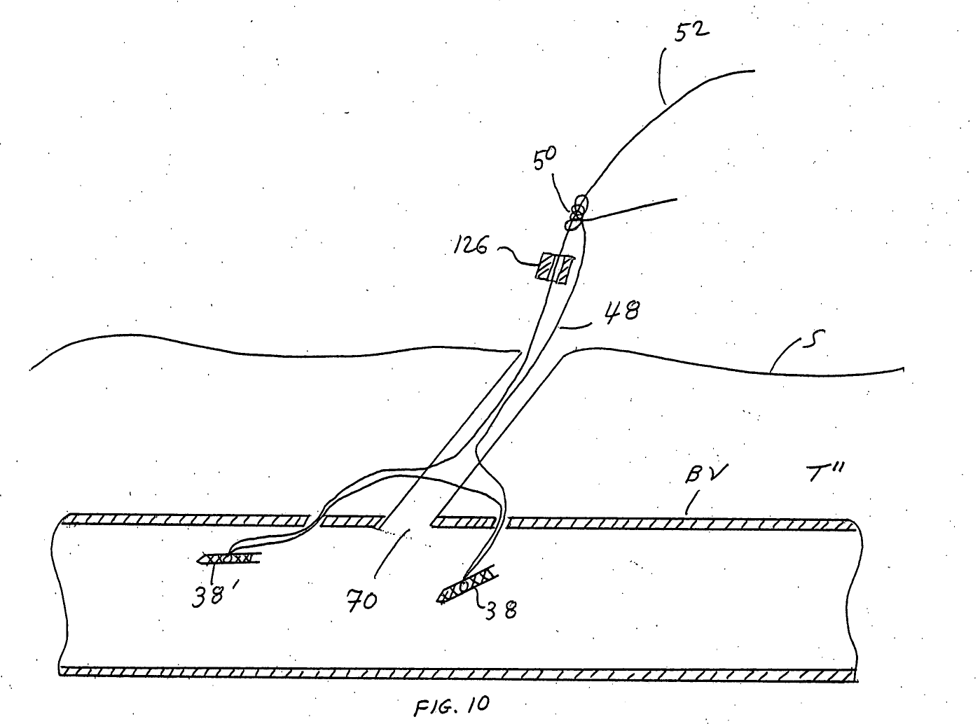

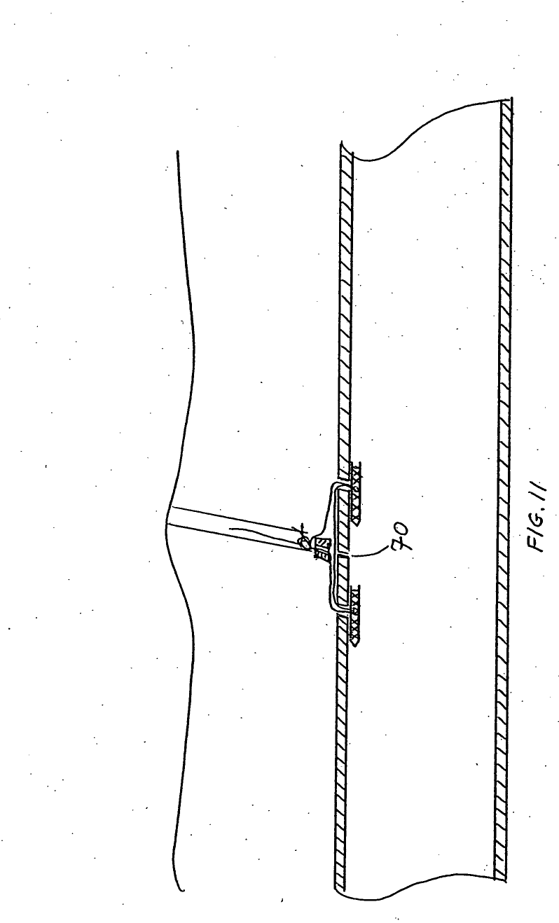

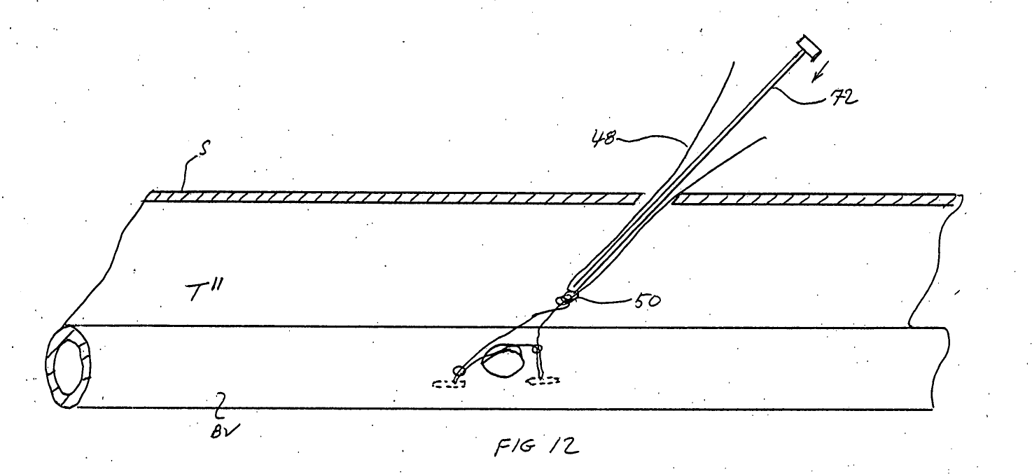

After a percutaneous procedure is completed, the instrument used to perform the procedure is generally removed, leaving indwelling guidewire 66 in place. The guidewire passes from the inside of the blood vessel BV, through the tissue T″ and out through the skin S. Device 10 is inserted into vessel BV by passing guidewire 66 through guidewire lumen 30 and sliding device 10 down over the guidewire into the blood vessel. Once device 10 is resident in the blood vessel, guidewire 66 can be withdrawn and discarded, leaving device 10 in place. The physician knows device 10 is properly located when blood is seen at the proximal end of indicator lumen 20. He then puts two fingers under shoulder 22 and, with his thumb, pushes handle 34 distally. In so doing, he causes the pushrods 32 and 32′ to slide distally through the pushrod lumens 14 and 14′ respectively. The pushrods, in turn, push on the anchors, forcing them out of recesses 74 and 74′ through holes 18 and 18′, thereby permitting barbs 46 and 46′ to deploy out away from the sides of the anchors. As the physician continues to push on handle 34, anchors 38 and 38′ are pushed into the walls of the blood vessel BV as seen in FIG. 9 and eventually through those walls. In this embodiment the anchors exit from their recesses at an oblique angle to catheter 12 in order to facilitate capture of the wall of the vessel BV on opposite sides of and adjacent puncture 70. Once the anchors are inside blood vessel BV, device 10 is removed, leaving the anchors in place, as shown in FIG. 10. Barbs 46 and 46′ prevent the anchors from inadvertently being withdrawn from the blood vessel. The physician then pulls on suture end 52 to pull anchors 38 and 38′ toward one another, optionally by use of pusher 72, thereby closing puncture 70 as shown in FIG. 11. The suture is then tied and left in place. Since the suture and the anchors are preferably made of resorbable material, after the normal bodily processes have sealed puncture 70, the suture and the anchors will be resorbed, leaving no foreign matter either in the blood vessel or in the tissue.

If embodiment of FIG. 7A or 7B is employed, instead of tying the suture, ring 196 is crimped to secure the anchor in place.

Although it is preferred that the anchors be provided with barbs to prevent accidental withdrawal, it is believed that barbs are not essential. The retraction prevention means in such an embodiment would comprise means for causing the anchor to turn sideways once it is inside the blood vessel. This can be accomplished by locating the suture mounting means so that when tension is applied to the suture the anchor is caused to turn sideways, thereby preventing accidental withdrawal.

If it is desired, suture 48 can be threaded through a plug 126 between slip knot 50 and one of the anchors. In this embodiment, when the suture is tightened, plug 126 is pushed down along the suture until it reaches the outside wall of the blood vessel BV, thereby covering puncture 70. Plug 126, like the other components left in the body, is preferably made of resorbable material.

As those skilled in the art will appreciate, the device of FIG. 1 need not have two pushrod lumens and two anchor recesses. Rather, it can have as few as one of each but preferably two or more. When two or more are employed, they are preferably located equidistant around catheter 12, each of which would operated precisely as described above. In addition, the device may have two or more anchors, but only one pushrod lumen and one anchor recess. In that event, two or more anchors would be loaded one behind another in the one pushrod lumen or, alternatively they could be loaded in a cartridge that is then loaded into the pushrod lumen. The pushrod would then eject one anchor and cause it to puncture the blood vessel wall. Device 10 would then be rotated, and the procedure repeated with the second anchor and so on until the physician is satisfied that sufficient anchors are in place to close the puncture properly. In such an embodiment, only one pushrod would be needed.

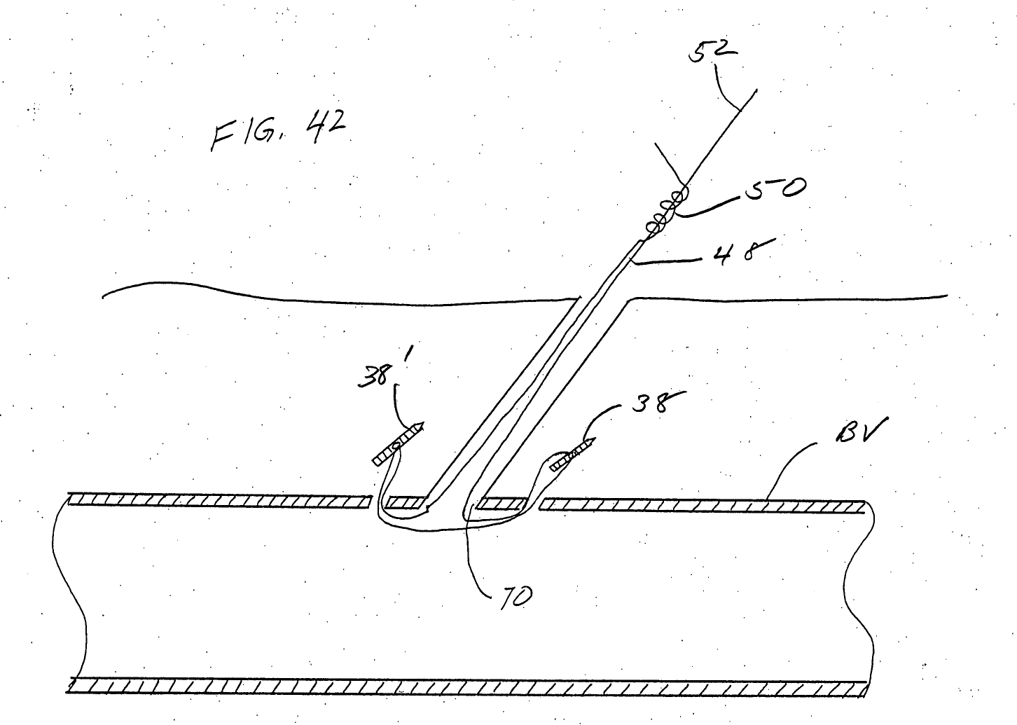

The embodiment of FIGS. 40-42 is similar to the embodiment of FIG. 1 except that in the FIG. 1 embodiment the anchors pass through the wall of the internal structure from the outside to the inside with the anchors being left inside the structure. In the embodiment of FIGS. 40-42, the anchors pass through the wall structure from the inside to the outside with the anchors being left outside the structure.

The device 10 of the embodiment of FIG. 40 is comprised of an elongated insertion tube 204 which has a guide wire lumen 30 at its distal end and a handle 36 at its proximal end. Intermediate between its proximal end and its distal end tube 204 is provided with anchor recesses 214 and 214′. As has been describe with respect to the prior embodiment, additional anchor recesses may be provided around the periphery of tube 204. Resident in the anchor recesses 214 and 214′ are anchors 38 and 38′ respectively. Distal of the anchor recesses, tube 204 is provided with an internal recess 206 and passing through tube 204 from its proximal end to internal recess 206 is lumen 14. A pull rod 208 is provided that has at its proximal end a pull rod handle 34. At its distal end, pull rod 208 is attached to platform 210 and attached to platform 210 are two pushrods 212 and 212′ which extend from platform 210 into the distal ends of anchor recesses 214 and 214′ respectively. Anchor recesses 214 and 214′ extend from internal recess 206 to the outer wall of tube 204. Finally, tube 204 is provided with an indicator lumen 20 that functions in the same way as the indicator lumen functions in the embodiment of FIG. 1.

Anchors of the embodiment of FIGS. 40-42 are substantially the same as the anchors of the embodiment of FIG. 1 and, as describe in connection with the embodiment of FIG. 1, they are mounted on a suture. Also as describe in connection with the FIG. 1 embodiment, the suture is provided with a slip knot 50 or other slide mechanism that permits tightening of the suture by pulling on the loose end that extends outside the body.

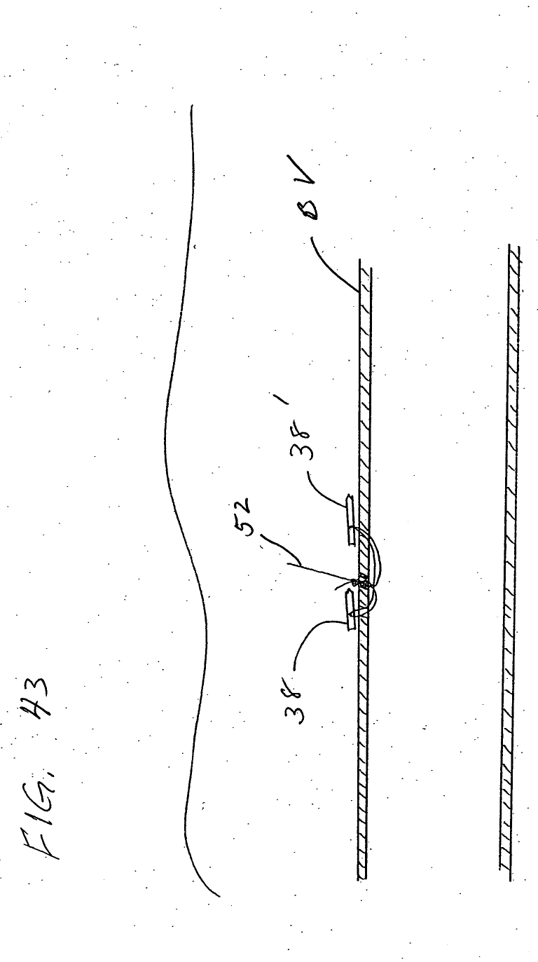

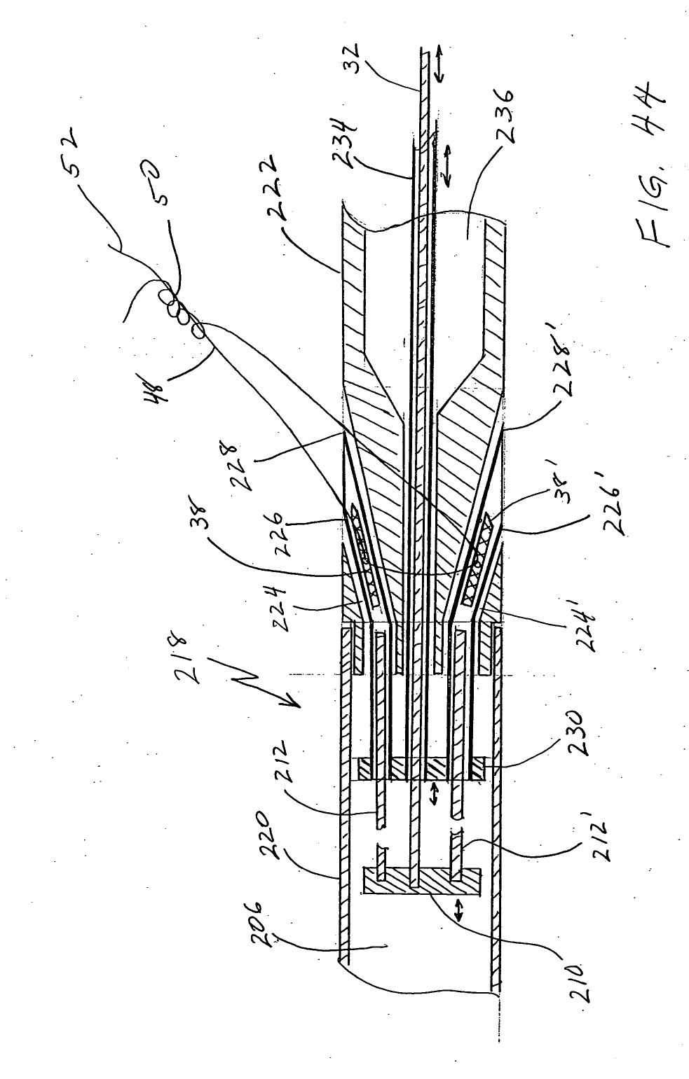

The device of FIG. 40 is inserted over a guide wire (not shown) into the target structure until fluid from the target structure enters the distal end 26 of indicator lumen 20 and is observed at the proximal end 28 of lumen 20. The operator then knows that the exit openings 216 and 216′ of the anchor recesses are inside the target structure. The guide wire is then preferably removed and force is exerted in the proximal direction on handle 34. This force causes platform 210 to move in a proximal direction, thereby forcing pushrods 212 and 212′ to slide proximally through anchor recesses 214 and 214′ respectively. Such proximal movement of pushrods 212 and 212′ forces anchors 38 and 38′ out of recesses 214 and 214′ through exit openings 216 and 216′ and through the wall of structure BV from the inside to the outside thereof as shown in FIG. 41. Distal force is then preferably exerted on handle 34 to make certain pushrods 212 and 212′ are retracted into tube 204. Tube 204 is then withdrawn, leaving anchors 38 and 38′ outside structure BV, as best seen in FIG. 42. The operator then pulls on suture 52 to bring the anchors toward one another, thereby closing wound 70, as best seen in FIG. 43. Unlike the embodiment of FIG. 1, when the embodiment of FIG. 40 is employed, nothing is left inside the blood vessel or other structure being repaired except the suture itself.

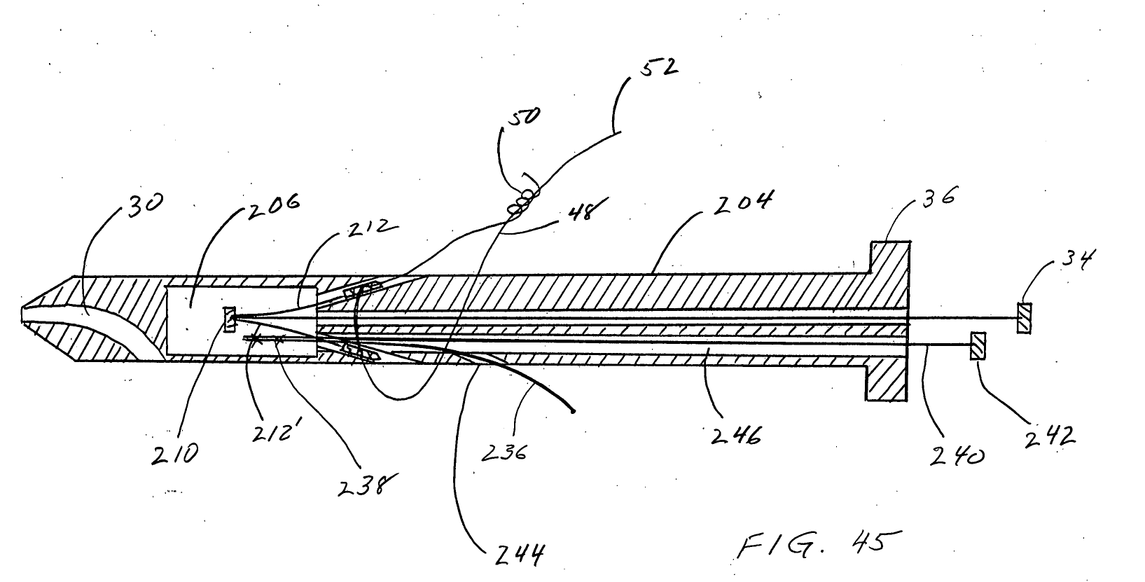

The embodiment of FIG. 44 is similar to the embodiment of FIG. 40 except that in the FIG. 44 embodiment, needles are used to puncture the wall of the blood vessel or other internal structure. Insertion tube 218 is similar to insertion tube 204 in that it has a guide wire lumen at the from end (not shown) and an internal recess 206. Although the embodiment of FIG. 44 is not shown with an indicator lumen, one similar to the one of the FIG. 40 embodiment could easily be incorporated. Insertion tube 218 is comprised of a distal section 220 and a proximal section 222. Recess 206 is located in distal section 220. At its distal end, section 222 is provided with two needle lumens 224 and 224′ which open at their distal ends in internal recess 220 and at their proximal ends open through the side wall of section 222. Housed within lumens 224 and 224′ are hollow needles 226 and 226′ having points 228 and 228′ respectively. Needles 226 and 226′ are attached at their distal ends to needle platform 230 housed within recess 206.

Section 222 of tube 218 is also provided with a through lumen 232 and passing through lumen 232 is hollow pull tube 234 which is attached at its distal end to needle platform 230. A handle (not shown), similar to handle 34 of the embodiment of FIG. 40 is affixed to the proximal end of pull tube 234.

Also resident in internal recess 206 is anchor platform 210. Attached at its distal end to platform 210 is pull rod 32 which passes through the lumen of hollow tube 234. Rod 32 is affixed at its proximal end to another handle, similar to handle 34. Also attached to platform 210 are pushrods 212 and 212′ which are sized to slide within the lumens of needles 226 and 226′ respectively. Finally, housed within needles 226 and 226′ are anchors 38 and 38′. In this embodiment, the needles act as the anchor recesses. As shown, section 222 can be hollowed out, as at 236 to provide increased flexibility for insertion tube 218.

Insertion tube 218 is preferably inserted over a guide wire until needle points 228 and 228′ are inside the blood vessel or other internal structure. While holding insertion tube 218 in place, hollow needle pull rod 234 is pulled in a proximal direction causing needles 226 and 226′ to emerge from lumens 224 and 224′ and to puncture the wall of the internal structure from the inside out. After points 228 and 228′ of needles 226 and 226′ have passed through the wall of the structure, anchor pull rod 32 is pulled in a proximal direction causing push rods 212 and 212′ to slide within the lumens of needles 226 and 226′, thereby forcing anchors out of needles 226 and 226′ so they are outside the wall of the internal structure. The rest of the procedure is as has been described with respect to the embodiment of FIG. 40.

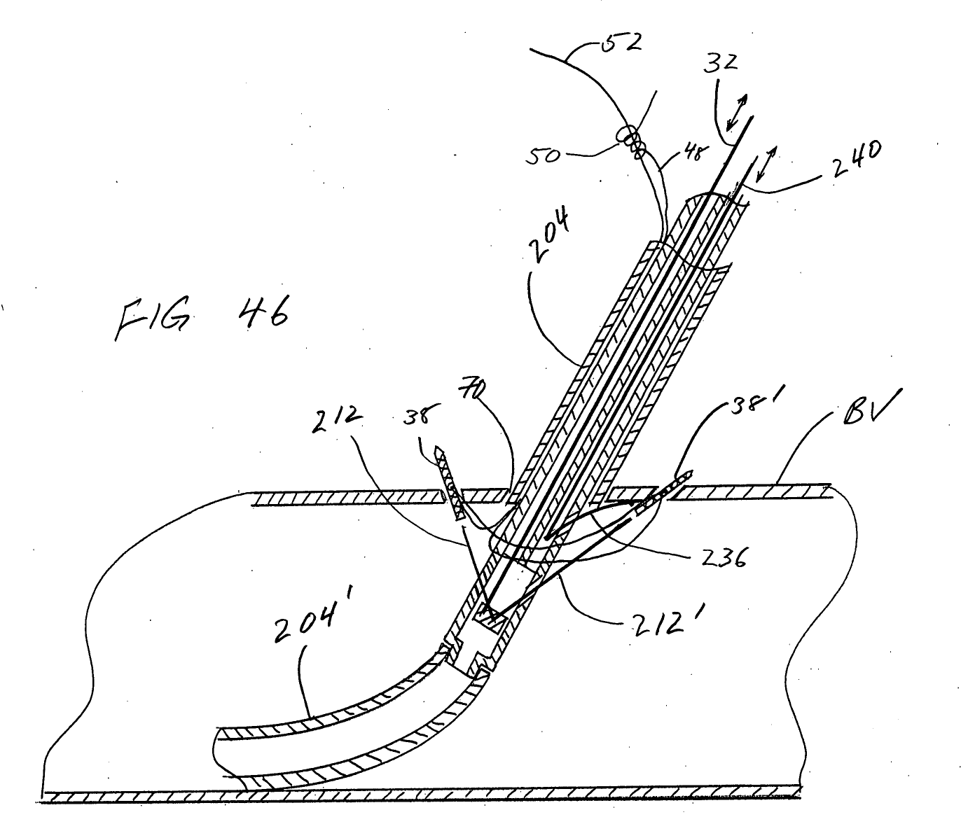

Yet another embodiment similar to that of FIG. 40 is shown in FIGS. 45 and 46. In this embodiment, the indicator lumen has been eliminated and replaced by a retractable locating barb 236 which is attached at its distal end 238 to operating rod 240. At its proximal end rod 240 is attached to handle 242. Rod 240 is housed in lumen 246 of insertion tube 204 and can move proximally and distally therein. When handle 242 is moved distally, barb 236 can be retracted into the body of insertion tube 204. When handle 242 is moved proximally, barb 236 is caused to exit tube 204 through opening and protrudes therefrom. In use, this embodiment can be inserted over a guide wire with barb 236 protruding. As tube 204 is inserted and opening 244 enters internal structure BV through wound 70, protruding barb 236 splays out so that when the operator exerts proximal pressure on tube 204, barb prevents retraction, as can best be seen in FIG. 46. The resistance caused by the splayed barb 236 signals to the operator that tube 204 is properly located. Alternatively, tube 204 can be inserted with the barb fully retracted in tube 204. The operator would then extend barb 236 by pulling on rod 240 and would then try to retract tube 204. If it retracts easily, the operator knows tube 204 is not far enough into the body. The barb would then be retracted, tube 204 inserted further into the body and the barb extended once again. By this trial and error method, positive location of the tube can be achieved.

With tube 204 properly located and barb 236 extended and splayed outwardly, the operator would pull on handle 34 to cause anchors 38 and 38′ to exit their respective recesses and pass through the wall of the internal structure, as described above in connection with the embodiment of FIG. 40. Once the anchors have passed through the wall of the structure, a distal force is applied to handle 34 to retract pushrods 212 and 212′, also as described in the discussion of the FIG. 40 embodiment. Handle 34 can be configured to overlay handle 242 so that when handle 34 is move distally, it causes handle 242 to move distally as well. In this way, moving handle 34 distally causes retraction, not only of pushrods 212 and 212′ but of barb 236 as well. Alternatively. Handles 34 and 242 can be made to operate independently.

As seen in FIG. 46, tube 204 can be made in two parts, similar to the two-part construction shown in FIG. 43. One advantage of this two part construction is that the distal end 204′ of the tube can be made to be more flexible so as not to cause damage to the inside of the internal structure BV.

FIGS. 15-19 show another embodiment of the insertion device of the present invention in which the anchors are inserted independently of one another. The insertion device 10 of this embodiment like the embodiment of FIG. 1, has a guidewire lumen 30 and an indicator lumen 20 that serve the same purposes as the corresponding elements serve in the earlier embodiment. In addition, this embodiment also contains two pushrod lumens 86 and 86′. The embodiment of FIGS. 15-19 is preferably provided with two pushrods 88 and 88′. Alternatively, it can be provided with only one pushrod that performs its task in lumen 86 and is then removed and inserted into lumen 86′ to repeat the process. Pushrod lumens 86 and 86′ terminate in anchor recesses 98 and 98′ respectively.

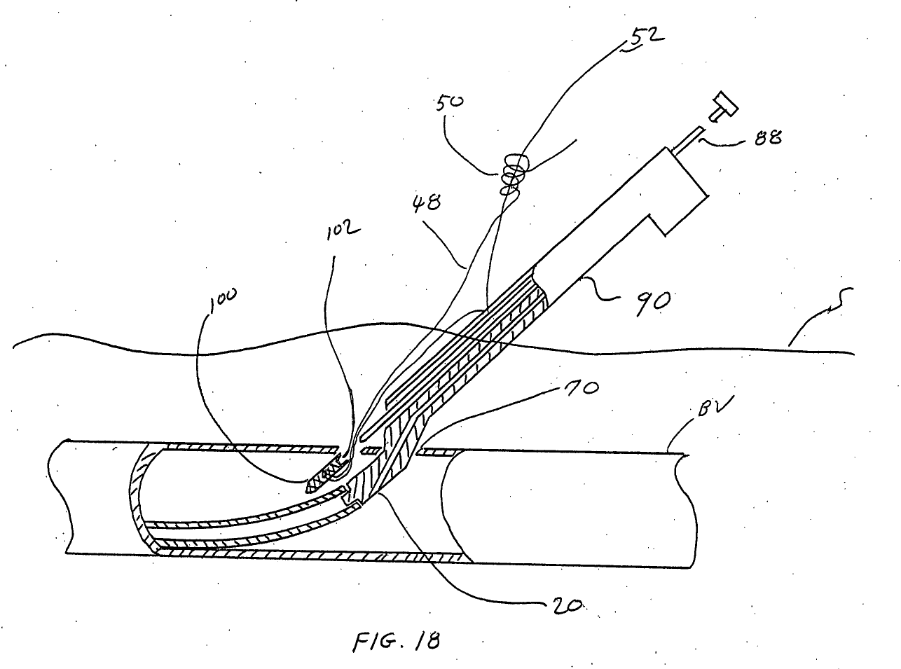

Catheter 90 of the embodiment of FIG. 16 has a proximal portion 92 which is radially offset from distal portion 94. The two portions are connected at transition zone 96. Pushrod lumens 86 and 86′ run from the proximal end 22 of catheter 90 to transition zone 96. Lumens 86 and 86′ (and, hence, anchor recesses 102 and 102′) terminate at their distal ends in openings 98 and 98′ respectively, both of which are in transition zone 96.

In addition, the embodiment of FIGS. 15-19, like previously described embodiments, contains an indicator lumen 20. At its distal end, indicator lumen 20 opens through the side of the distal portion of 94 of catheter 90, slightly distal of transition zone 96.

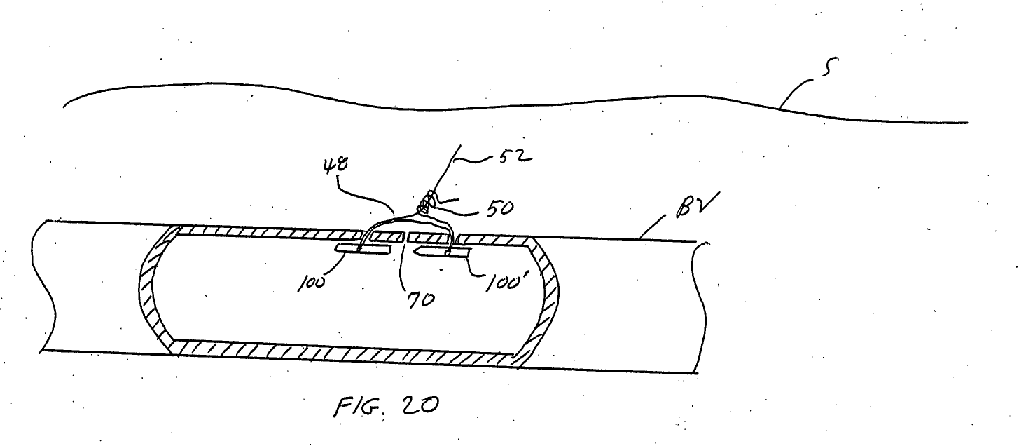

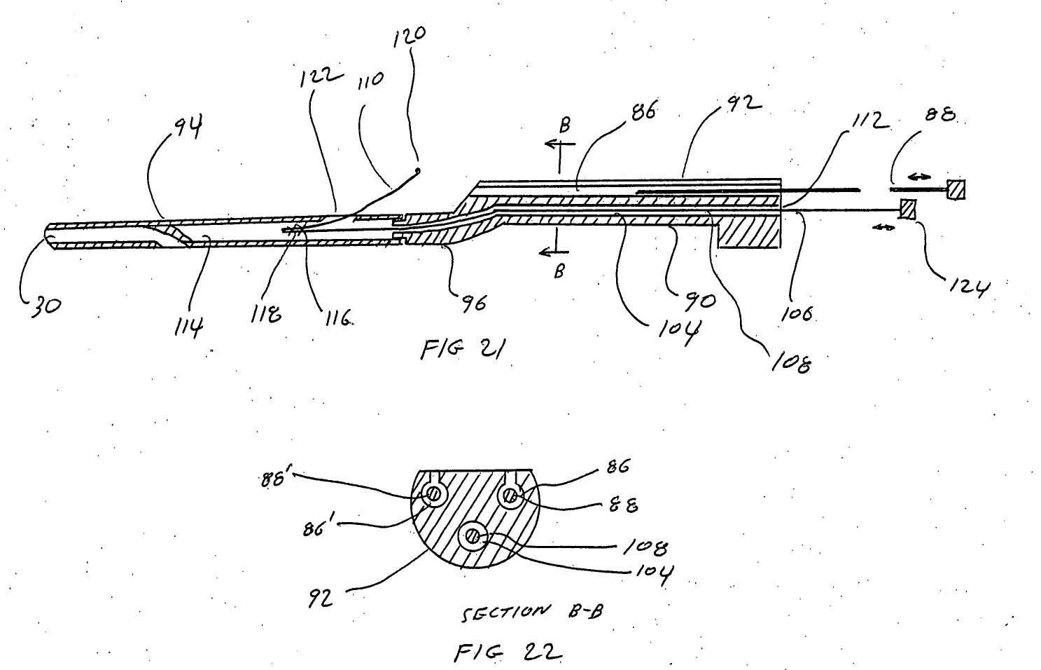

The embodiment of FIGS. 15-19 is employed by initially inserting anchors 100 and 100′ into their respective recesses 102 and 102′. The guidewire lumen of the device is then passed over an indwelling guidewire until blood is observed exiting from the proximal end of indicator lumen 98, and the guidewire is withdrawn, all as described above. Forward pressure is then exerted on pushrod 88 to force anchor 100 out of its recess 102 and through the wall of the blood vessel or other internal structure, as shown in FIG. 18. When anchor 100 is through the wall of the blood vessel, pushrod 88 is retracted into tube 90, leaving anchor 100 inside the lumen of the blood vessel. As described above, the anchor is preferably provided with a barb but it need not be. Tube 90 is then rotated, preferably about 180°, as shown in FIG. 19. Forward pressure is then exerted on pushrod 88′ to force anchor 100′ from its recess 102′ and into and through the blood vessel wall.

The device of the embodiment of FIG. 15 is then withdrawn and suture 48 is tightened by pulling on end 52, as described above. This pulls the anchors toward one another and closes the puncture 70 as shown in FIG. 20.

Although the embodiment of FIG. 15 is illustrated as having two pushrod lumens, it may have more or less, as may be deemed appropriate. If three or more pushrod lumens are employed, they would preferably be located equidistant around catheter 90 and when used, the physician would rotate catheter 90 so as to place each pushrod lumen seriatim in position to insert the anchors where it is deemed best that they be placed.

Alternatively, the embodiment of FIG. 15 could be made with only one pushrod lumen and one anchor recess opening, as describe above with respect to the embodiment of FIG. 1. As yet another alternative, it could have two pushrod lumens but only one anchor recess opening. One of the pushrod lumens would then not communicate directly with its own recess opening but, instead, at its distal end, would communicate with the first pushrod lumen. The second pushrod lumen would then act as a holder for additional anchors. At the distal end of that second pushrod lumen there would preferably be a spring loading mechanism urging the bottom most anchor into the anchor recess at the distal end of the first pushrod lumen. In use, the first anchor would be pushed into the blood vessel, as described above, the pushrod would be withdrawn far enough to permit the spring loading mechanism to push the next anchor into the anchor recess of the first pushrod lumen and the procedure would be repeated. This could be done until all anchors have been ejected.

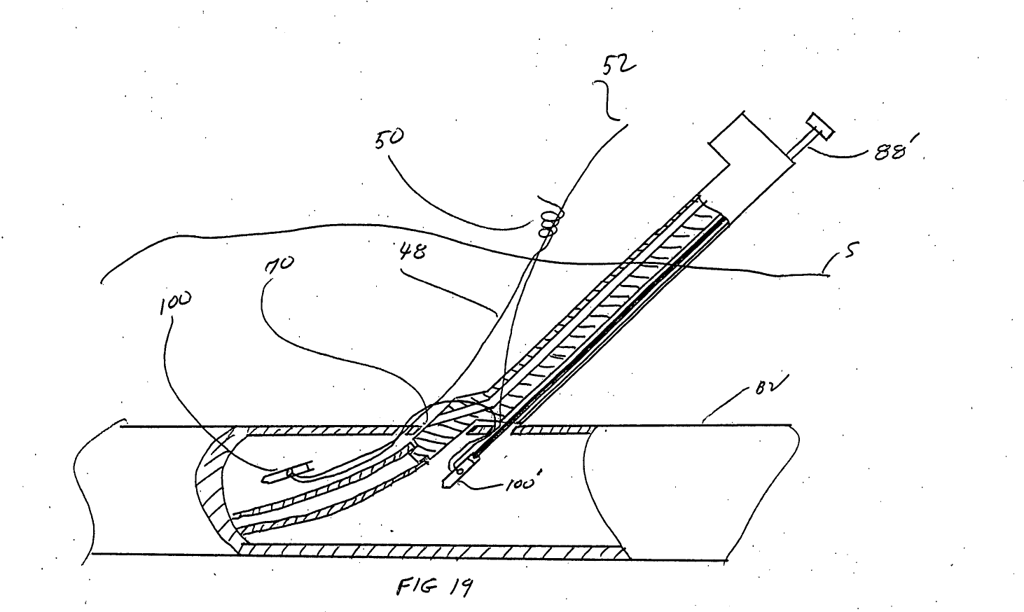

While the anchor recesses have been described as being fully enclosed, that is not necessary. Rather, as shown in FIG. 17, their sides are preferably open, through slots 128 and 128′, to the exterior of catheter 90 (or to the exterior of catheter 20 of the FIG. 1 embodiment). These slots permit the sutures to hang freely outside the tube even while the anchors are within their recesses so that the suture need not be squeezed into the recesses with the anchors, thereby risking damage to the sutures or binding thereof. In order that the anchors remain in their recesses until needed, slots 128 and 128′ should be smaller than the diameter of the anchors. Similarly, the pushrod lumens can be open, through slots (not shown), to the exterior of the catheter.

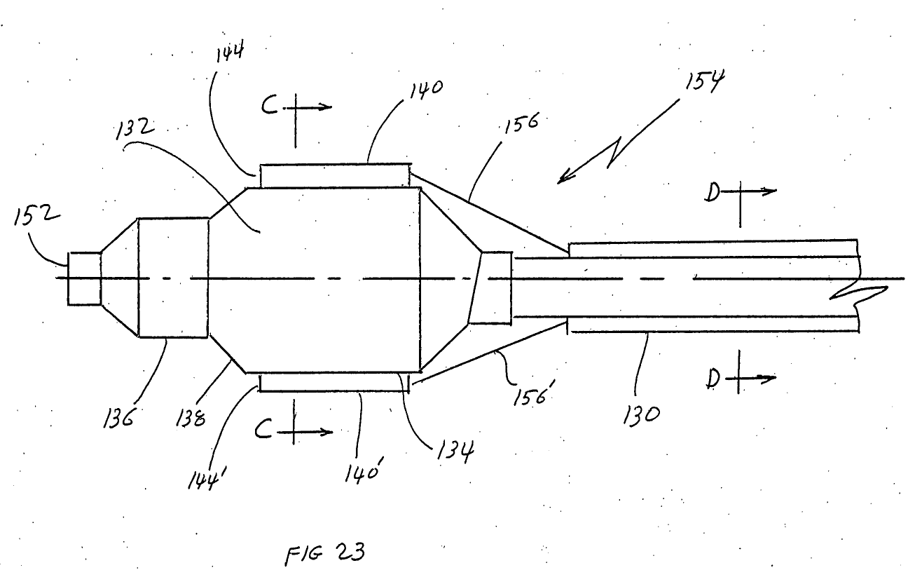

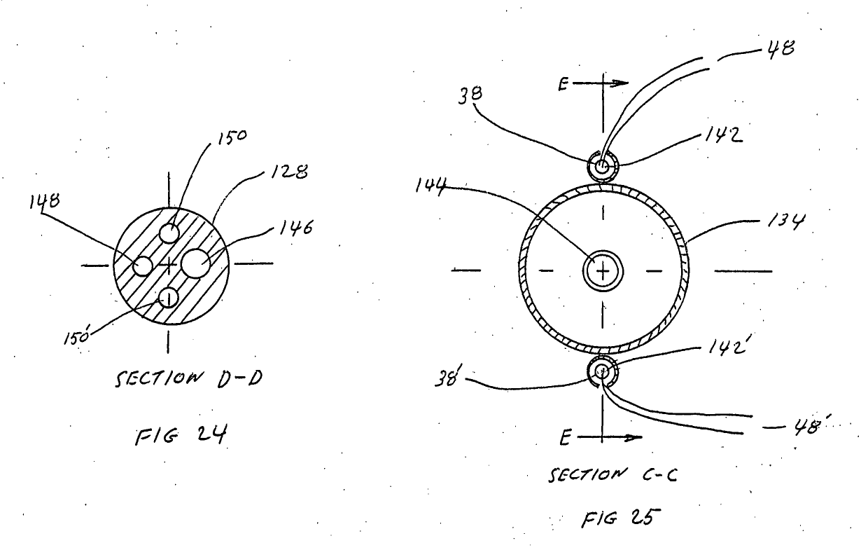

FIGS. 21-22 depict another embodiment of the instant invention. The embodiment of FIGS. 21 and 22 is designed primarily for suturing an internal structure that is either constantly moving or sufficiently flexible so that it would move out of the way if an attempt were made simply to force an anchor through it without somehow stabilizing it. The structure of the FIG. 21 embodiment is very similar to that of the structure of the FIG. 15 embodiment. In this embodiment, device 10 is comprised of a catheter 90 having proximal portion 92 and distal portion 94 radially offset from one another. The two portions are joined in transition zone 96. Like the previous embodiments, this one also has a guidewire lumen 30 but does not have a indicator lumen. It also has two anchor pushrod lumens 86 and 86′ and two anchor pushrods 88 and 88′ as well as anchor recesses 102 and 102′ and anchors 100 and 100′, all as described in connection with the FIG. 15 embodiment. In addition, the embodiment of FIGS. 21 and 22 has a stabilizer lumen 104 to accommodate stabilizer 106 that has a pushrod section 108 and an anchor section 110. Stabilizer lumen 104 has a distal end 114 and a proximal end which terminates in opening 112. Intermediate between the proximal and distal ends of stabilizer lumen 104 is a side opening 122. Distal end 116 of stabilizer pushrod section 108 is attached to distal end of stabilizer anchor 110. Stabilizer anchor 110 is preferably made of flexible stainless steel so that it can lay substantially flat against the side of pushrod section 108 when the distal end of pushrod section 108 and distal end of stabilizer anchor 110 are pushed into distal end 114 of stabilizer lumen 114. Stabilizer anchor is spring loaded so that when no external forces are exerted on it, its proximal end is deployed spaced apart from the pushrod section 108, as seen in FIG. 21. Finally, stabilizing pushrod section 108 is attached at its proximal end to operating handle 124.

When prepared for use, the embodiment of FIGS. 21 and 22 has the two suture anchors 100 and 100′ (not shown) resident in their respective recesses 102 and 102′ (not shown) as was discussed above with respect to the embodiment of FIG. 15 and both anchors are mounted on suture 48 also as discussed above. In addition, pushrods 88 and 88′ are partially inserted into their respective lumens 86 and 86′ so that they are positioned to push anchors 100 and 100′ out of their respective recesses. Stabilizer 106 is resident in its lumen 104 with handle 124 having pushed stabilizer 106 distally until stabilizer anchor 110 is fully within lumen 104. Using the guidewire lumen 30, and preferably guided by use of fluoroscopy, ultrasound imaging or some other visualizing technique, the device is then threaded over an indwelling guidewire until distal opening 86 of recess 102 is juxtaposed the section of the structure to be sutured, for example, one of the leaflets of the mitral valve, with opening 122 distal to said leaflet or other structural element. Stabilizer handle 124 is then pulled in a proximal direction, thereby causing the proximal tip 120 of stabilizing anchor 110 to exit opening 122. As anchor tip 120 continues to move in a proximal direction, it encounters and then perforates the leaflet. With the leaflet impaled on stabilizer anchor 110, further movement of the leaflet is prevented. While stabilizing anchor 110 holds the leaflet in place, pushrod 88 is extended distally, thereby forcing anchor 100 out of recess 102 and through the leaflet. Pushrod 88 is then withdrawn into its lumen and stabilizer handle 124 is pushed distally to force distal ends 116 and 118 of the stabilizer pushrod and anchor sections, respectively, into the distal end 114 of stabilizer lumen 104. This causes tip 120 of stabilizer anchor 110 to release the leaflet, leaving only suture anchor 100 on the distal side of the leaflet with suture 48 passing through the perforation made by anchor 100. Device 10 is then rotated so that the opening of recess 102′ is juxtaposed the second structure to which the first structure is to be sutured, for example, a second leaflet of the mitral valve. The process is then repeated so that suture anchor 100′ is on the distal side of the second leaflet, with the suture passing through the perforation made in that second leaflet by anchor 100′. End 52 of the suture is pulled tight to bring the two anchors close together and a knot is tied to prevent slipknot 50 from loosening. In this embodiment, it is preferable that the suture and the suture anchors not be made of resorbable material. For this embodiment, the anchors may be made of metal, such as nitinol and stainless steel, or some biocompatible plastic that is not resorbable.

As will be apparent to those of skill in the art, the suture anchor and the stabilizing anchor can be reversed. Thus, the stabilizing anchor 110 could be attached at its proximal end to the pushrod section 108 of stabilizer 106 and the stabilizing anchor would then be deployed by pushing the stabilizer in the distal direction. The suture anchors would then be housed in recesses in the distal end 114 of stabilizer lumen 104 and the anchors would be deployed by pulling them proximally into the internal structure.

Device 154 of the embodiment shown in FIGS. 23-26 is intended for use, among other things, for anchoring prostheses like heart valves and valvuloplasty rings in place. It is comprised of a multi-lumen catheter 130, to the distal end of which is affixed a stepped balloon 132 having large diameter section 134 and a small diameter section 136 and a neck zone 138 between the two. Mounted on the outside surface of section 134 are anchor guides 140 and 140′ having anchor recesses 142 and 142′ respectively therein. At the distal end of each of recesses 142 and 142′ are anchor ejection openings 144 and 144′.

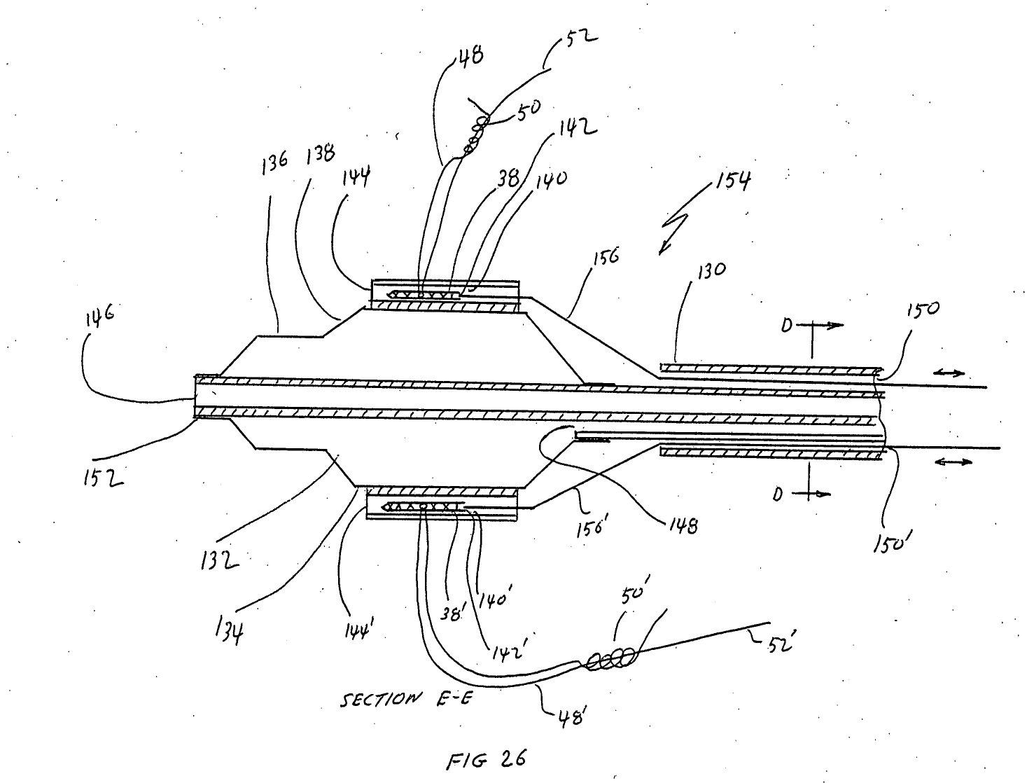

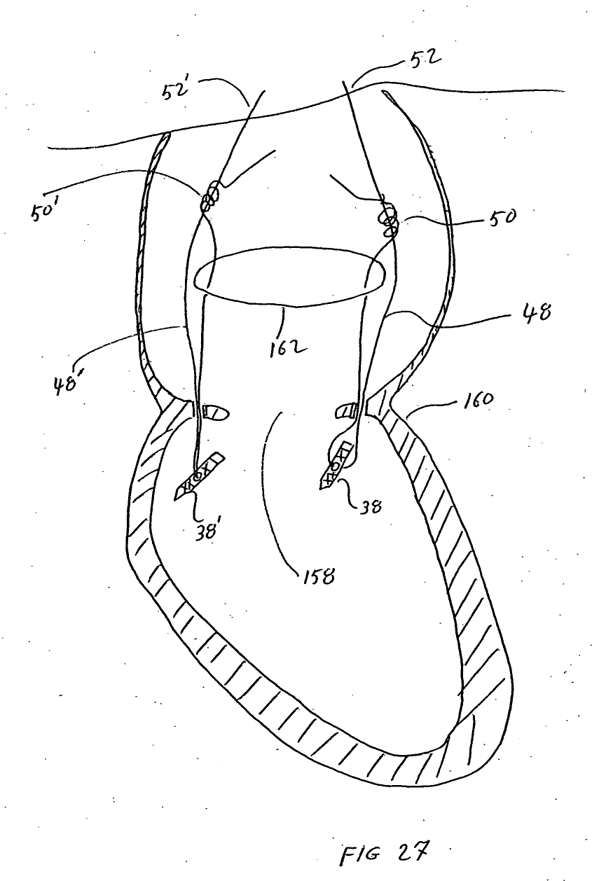

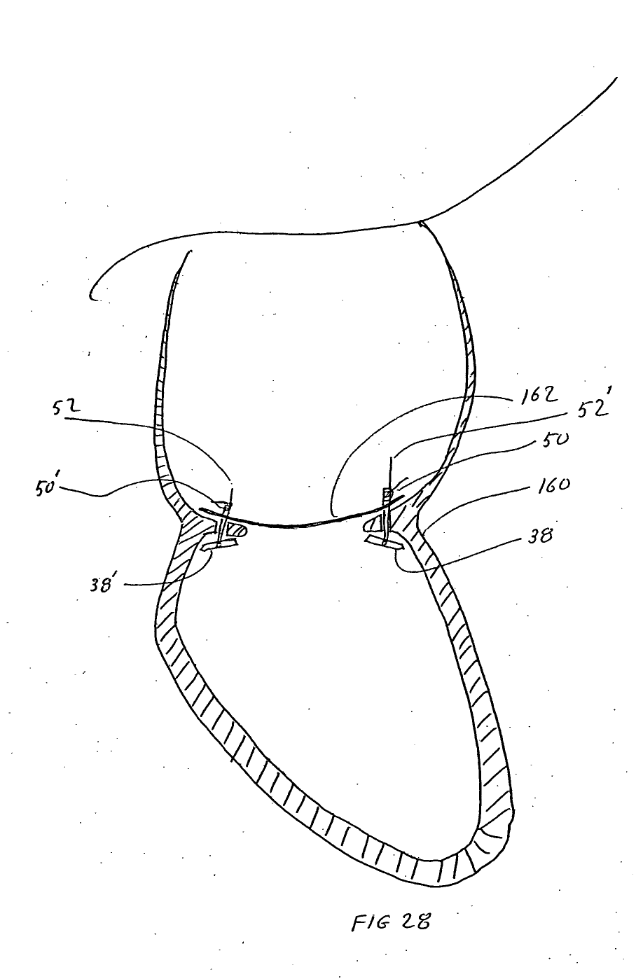

Catheter 130 contains a guidewire lumen 146, an inflation lumen 148 and two pushrod lumens 150 and 150′. As can be seen from FIG. 26, guidewire lumen passes through balloon 132 and terminates at distal′ end 152 of device 154. In each of recesses 142 and 142′ is housed an anchor 38 and 38′ similar to the anchors described above. Anchors 38 and 38′ are attached to sutures 48 and 48′ respectively. Sutures 48 and 48′ are provided with slip knots 50 and 50′ respectively and suture ends 52 and 52′ respectively. In addition, device 154 of FIG. 23 is provided with two pushrods 156 and 156′. The pushrods begin, at their distal ends, in their respective anchor guides, and continue into and through their respective pushrod lumens in catheter 130, terminating in an operating handle (not shown) as has been describe above.

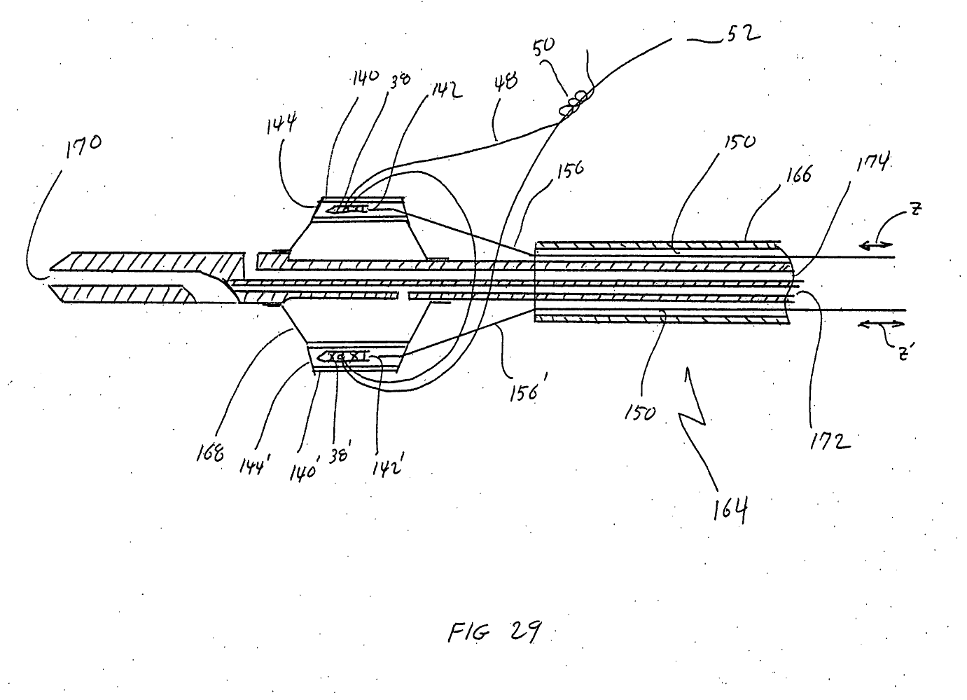

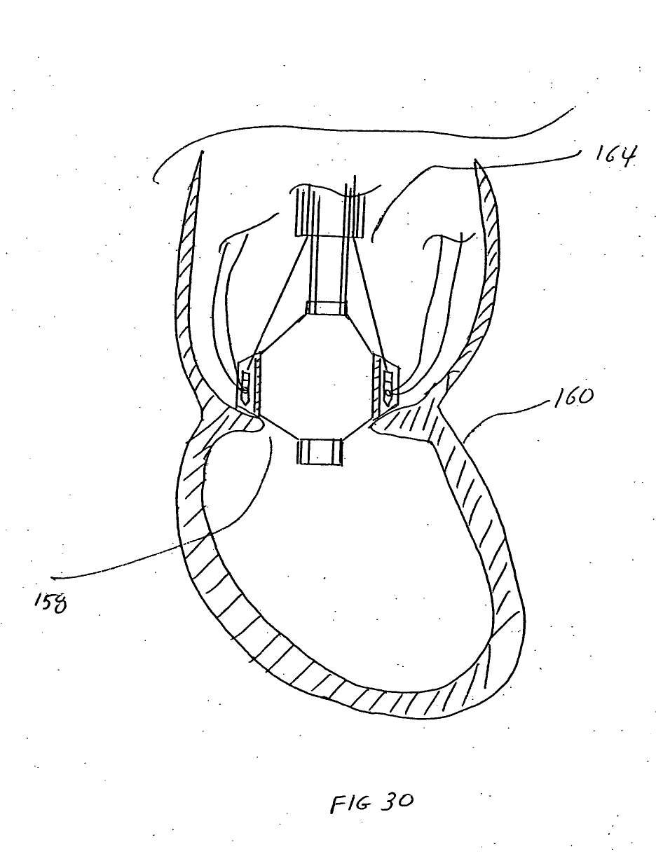

Device 154 is inserted into the target organ, for example, the heart, under the guidance of fluoroscopy, ultra sound imaging or some other visualizing technique, as is known to those of skill in the art. When it is properly located, for example, next to the mitral valve, as shown in FIG. 27, balloon 132 is inflated through inflation lumen 148. Because of the contour of the balloon, neck zone 138 seats against the annulus of the valve and is centered in valve opening 158, thereby assuring substantially equal tissue capture at all anchor sites at all points around the valve annulus. Force is then applied to the proximal end of pushrods 156 and 156′ to cause them to move distally in their respective pushrod lumens. As the pushrods move distally, they force the anchors 38 and 38′ out of recesses 142 and 142′ respectively through ejection openings 144 and 144′. As they exit from their recesses, anchors 38 and 38′ are forced through wall 160 of the annulus of the valve. Once balloon 132 has been deflated, device 154 can be withdrawn leaving only the anchors and their associated sutures in place, as can be seen in FIG. 28, with the sutures being attached to the anchors and passing out of the body so the physician can tighten up on them individually.

When the device of FIG. 23 is used to anchor a prosthesis, for example, an annuloplasty ring, a valvuloplasty ring or a heart valve, the prosthesis is preferably mounted on the sutures 48 and 48′, as is shown, for example, in FIG. 7B, prior to insertion into the body. The prosthesis, like annuloplasty ring 162, is held on the sutures between the slip knots 50 and 50′ and the anchors 38 and 38′, as can best be seen in FIG. 27. After the anchors are in place and device 154 has been withdrawn, the physician can then tighten up on each suture individually by pulling on ends 52 and 52′, thereby anchoring the ring in place, as can be seen in FIG. 28. Two separate sutures are employed in this embodiment because the object here is not to bring two sides of an opening together, as in the prior embodiments, but to anchor a prosthetic device in place without pulling the valve opening or other structure closed.

Device 154, as well as devices 164 and 176 below, can be used to suture a mesh-like material, like a DACRON patch, over a puncture or over a weakened portion of an internal structure to provide reinforcement or to repair an organ defect, for example an atrial septum defect.

As those of skill in the art will readily understand, the device of FIG. 23 need not have only two anchor guides, anchors and pushrods. Rather, as many anchor guides as may be needed can be mounted around the periphery of the balloon so that the physician can use as many anchors and sutures as the structure and prosthesis may need.

Although size is not a feature of the present invention, it is believed that section 136 of balloon 132 can be as small as 0.10″ in diameter and as large as about 2″ in diameter. Further, it is believed that section 134 of balloon 132 should be between about 0.10″ in diameter larger than section 136 and as much as about 0.30″ larger. The balloon sizes will be selected depending on the size of the targeted internal structure and the size of the prosthesis.

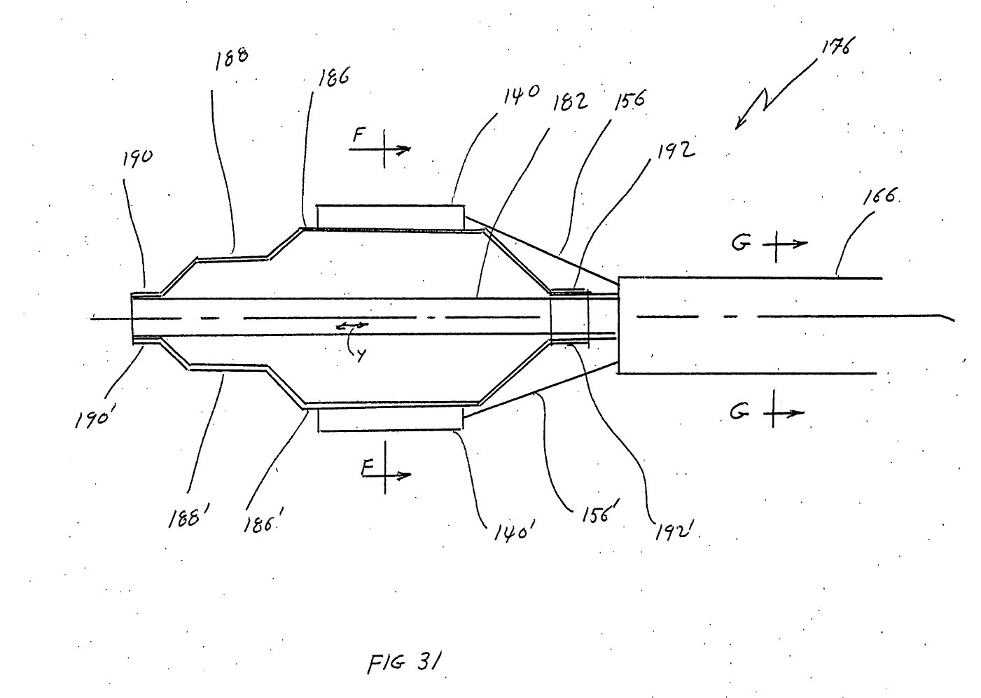

FIG. 29 shows an embodiment that is similar to that of FIG. 23, but is designed for smaller openings in internal organs than those for which the embodiment of FIG. 23 is primarily intended. Device 164 of FIG. 29 is comprised of a catheter 166 and a balloon 168. At the distal end of device 164 is a relatively short guidewire lumen 170 that does not go through catheter 166, similar to the guidewire lumen shown in FIG. 1. Catheter 166 has an inflation lumen 172, an indicator lumen 174 and pushrod lumens 150 and 150′. Inflation lumen 172 is used to inflate balloon 168 and indicator lumen 174 is used to alert the physician that device 164 is properly located, as describe in connection with the embodiment of FIG. 1. Since this embodiment is designed for relatively small openings or punctures, a stepped balloon is not needed.

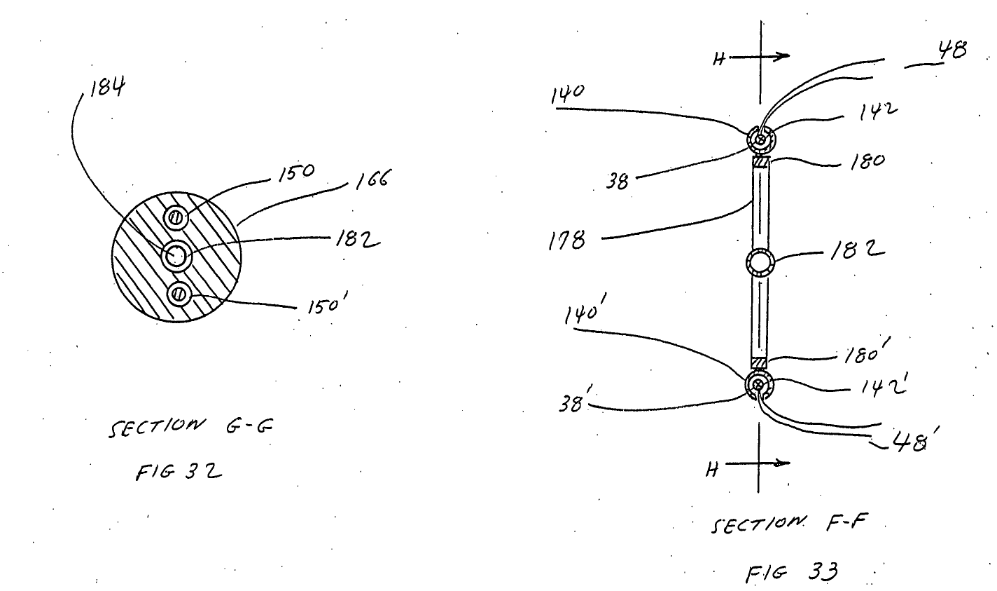

The FIG. 29 embodiment also has anchor guides 140 and 140′ in which there are located anchor recesses 142 and 142′ respectively. In addition, it also has pushrods 156 and 156′ that function as described in connection with the embodiment of FIG. 23. As described above, this embodiment may have more than two anchors, pushrods, anchor guides and anchor recesses as needed. When prepared for use, anchors 38 and 38′ reside in recesses 142 and 142′ respectively. Guidewire lumen 170 is passed over an indwelling guidewire until fluid emerges from the proximal end of indicator lumen 174, alerting the physician that balloon 168 is properly placed, as depicted in FIG. 30. As in the previous embodiments, the pushrods are then pushed distally, as indicated by double headed arrows Z and Z′ causing the anchors to exit through openings 144 and 144′ and through an adjacent wall of the internal structure. Balloon 168 is then deflated, device 164 is withdrawn and suture 48 is tightened as describe above to pull the anchors toward one another, thereby to close the puncture.

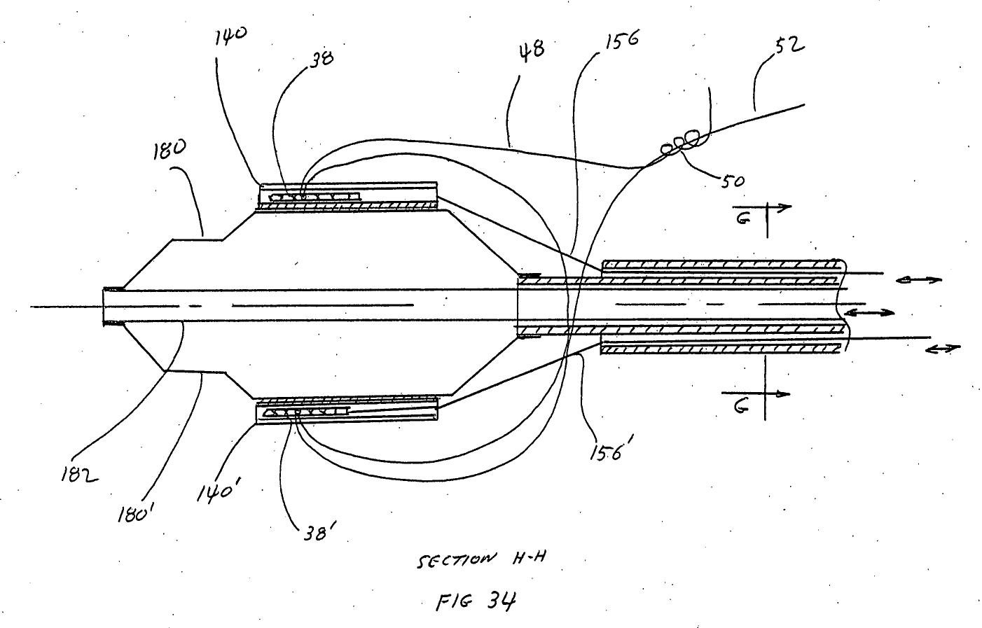

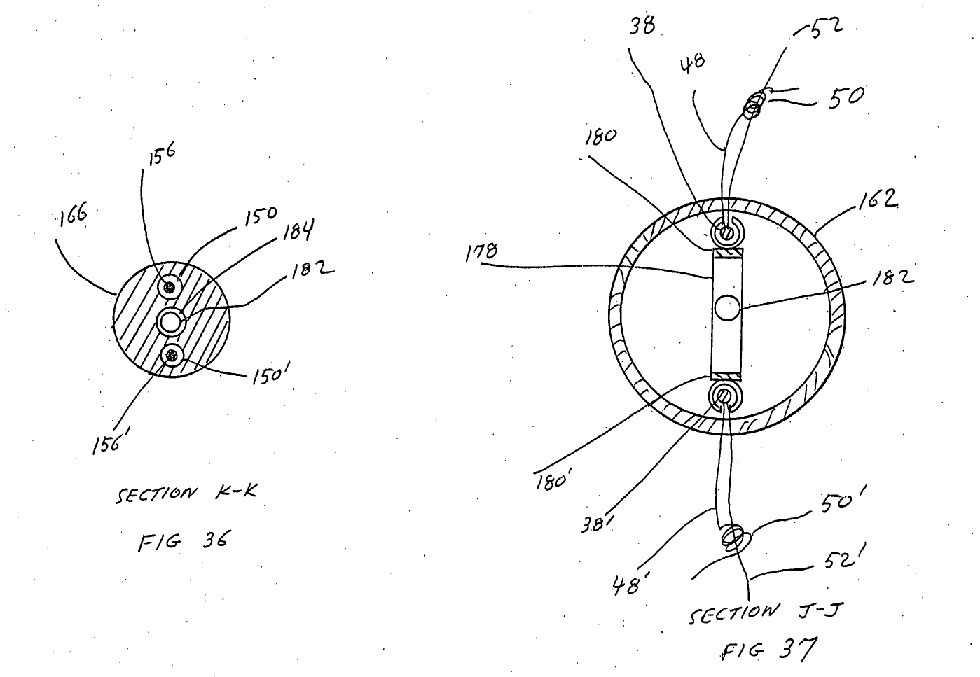

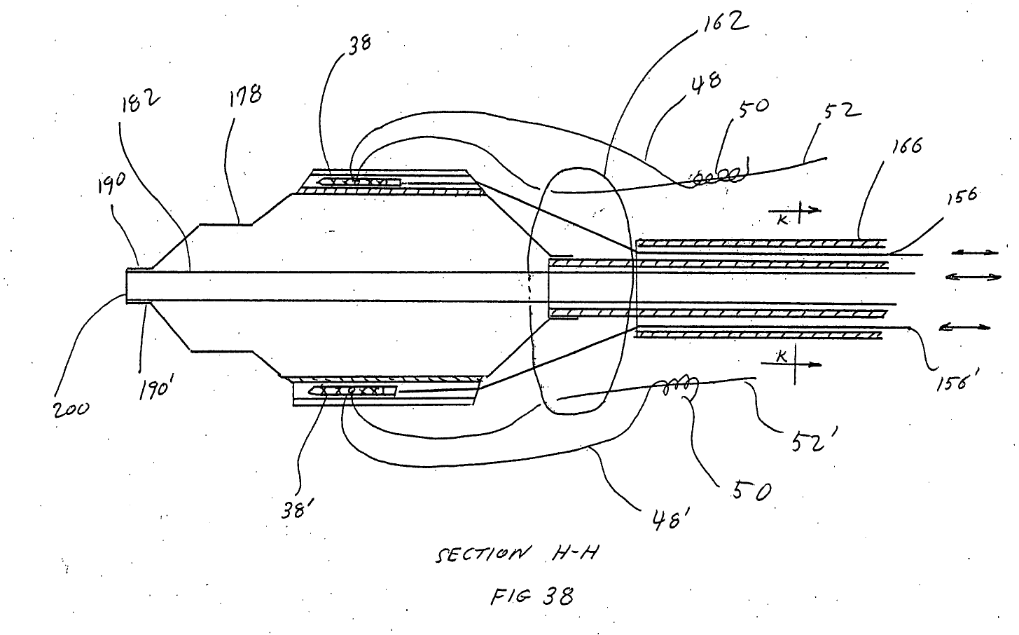

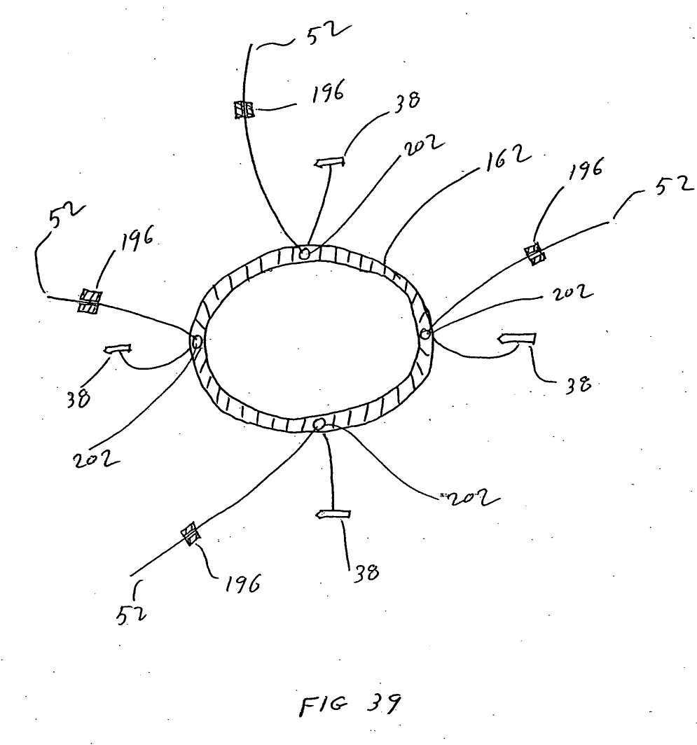

The embodiment shown in FIGS. 31-37 is similar to the embodiment of FIG. 23 except that the FIG. 31 embodiment employs a scaffold or web-like structure to support the anchor guides instead of the balloon of the FIG. 23 embodiment. Device 176 is comprised of a multi-lumen catheter 166 and a scaffold 178. Catheter 166 contains deployment lumen 194 and two pushrod lumens 150 and 150′. Scaffold 178 is comprised of support struts 180 and 180′ on which are mounted anchor guides 140 and 140′ respectively. Anchor recesses 142 and 142′ are within guides 140 and 140′ respectively. Housed within the recesses are anchors 38 and 38′ to which are attached sutures 48 and 48′ respectively. Pushrods 156 and 156′ pass through pushrod lumens 150 and 150′ respectively and into recesses 142 and 142′. In addition, passing through expansion catheter 166 is operating tube 182 that is movable distally and proximally, as indicated by double headed arrow Y, within deployment lumen 194. Passing through tube 182 is guidewire lumen 184. Depending on the application, struts 180 and 180′ can be shaped similarly to balloon 132, in that they may have a large diameter section 186 and 186′ as well as a smaller diameter section 188 and 188′. Alternatively, they may be shaped more like the balloon of FIG. 29 with only one section. Struts 180 and 180′ are made of a material, such as nitinol, that permits causing them to collapse when force is applied distally to end 200 and expanding them by releasing said distal force. Struts 180 and 180′ are affixed at their distal ends 190 and 190′ to tube 182 and at their proximal ends 192 and 192′ to catheter 166. FIG. 31 shows struts 186 and 186′ in their at rest position. The physician can cause them to collapse to a smaller size by pushing distally on operating tube 182 in order to facilitate passage through smaller lumens within the body.

Device 176 of FIG. 31 is deployed in the same way as device 154 of FIG. 23 except that, instead of the physician inflating a balloon, as in the FIG. 23 embodiment, here he first collapses struts 180 and 180′ until device 176 is properly located and then tube 182 is released enabling struts 180 and 180′ to resume their normal, at rest configuration.

This embodiment, like all the previously described embodiments, is not limited to using only two anchors. Rather, it can have as many or as few anchors as the physician believes necessary. Each anchor would be housed in its own anchor guide and each anchor guide would be mounted on its own strut. Thus, preferably, the scaffold would have an equal number of struts and anchors.

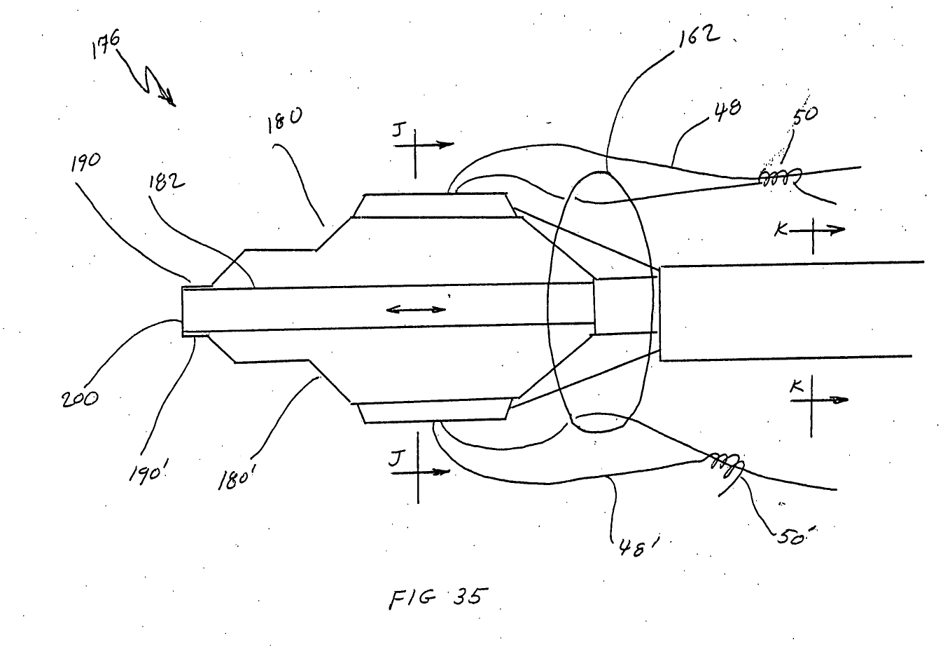

The device of embodiment of FIG. 31 (as is true of all the embodiments described herein) can have anchors 38 and 38′ mounted on a common suture 48, as shown in FIG. 34 or they can each be attached to their own individual sutures 48 and 48′, as shown in FIG. 35. The use of a single suture is believed to be more suitable for use in closing a puncture by pulling on the suture like a purse string. The use of individual sutures is believed to be more suitable for use in anchoring a prosthetic device, like the valvuloplasty ring 162, as depicted in FIG. 35.

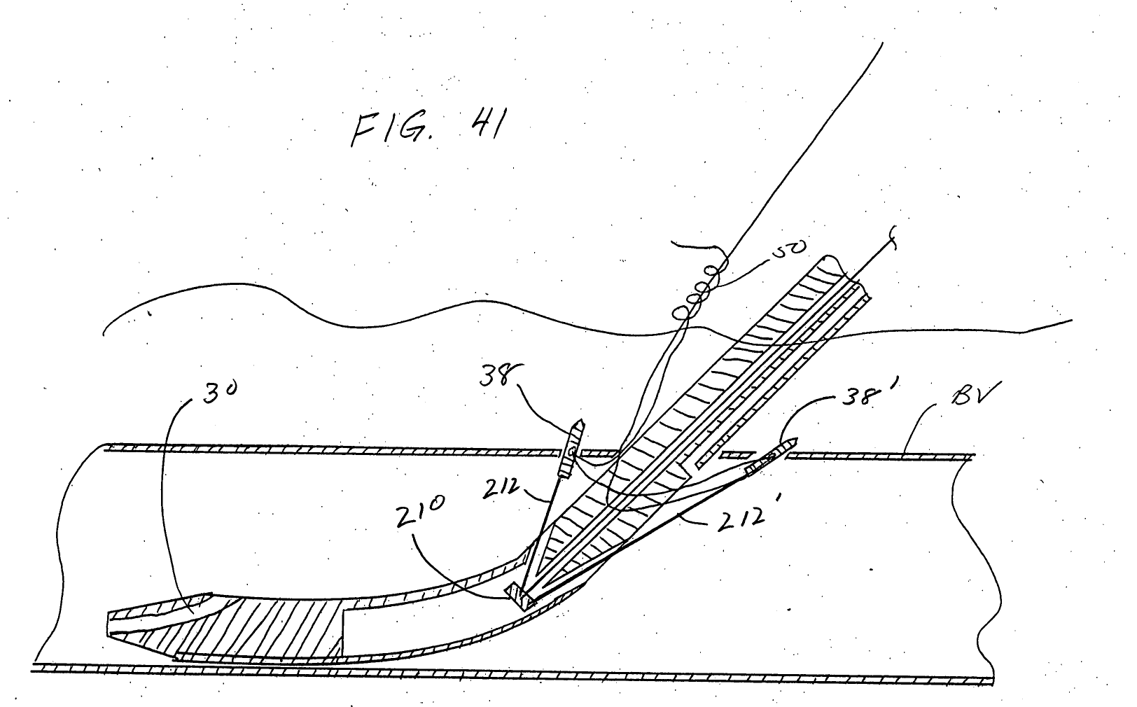

FIG. 39 depicts a valvuloplasty ring 162 having four suture holes 202 arranged around the periphery of the annulus of the ring. The ring shown in FIG. 39 is particularly suitable for use with a suturing device similar to those shown in FIGS. 23, 26, 29 or 31. Preferably, the suturing device used to insert the device of FIG. 39 will have four anchor guides, each anchor guide having an anchor recess for one of the anchors 38. Ring 162 is preferably made of deformable material, as those of skill in the art will recognize, to enable feeding it through the venal or arterial systems to the location where needed. Anchors 38 would then be ejected from their recesses and through the annulus of the valve seat. While the physician holds the suture ends 52, crimp ferrules 196 are slid down the suture until they seat against the face of the ring. Ferrules 196 are then crimped tight, thereby locking ring 162 in place. Of course, as those of skill in the art will readily appreciate, ring 162 could be held in place by more than four sutures or by fewer than four, all well within the scope of the present invention.

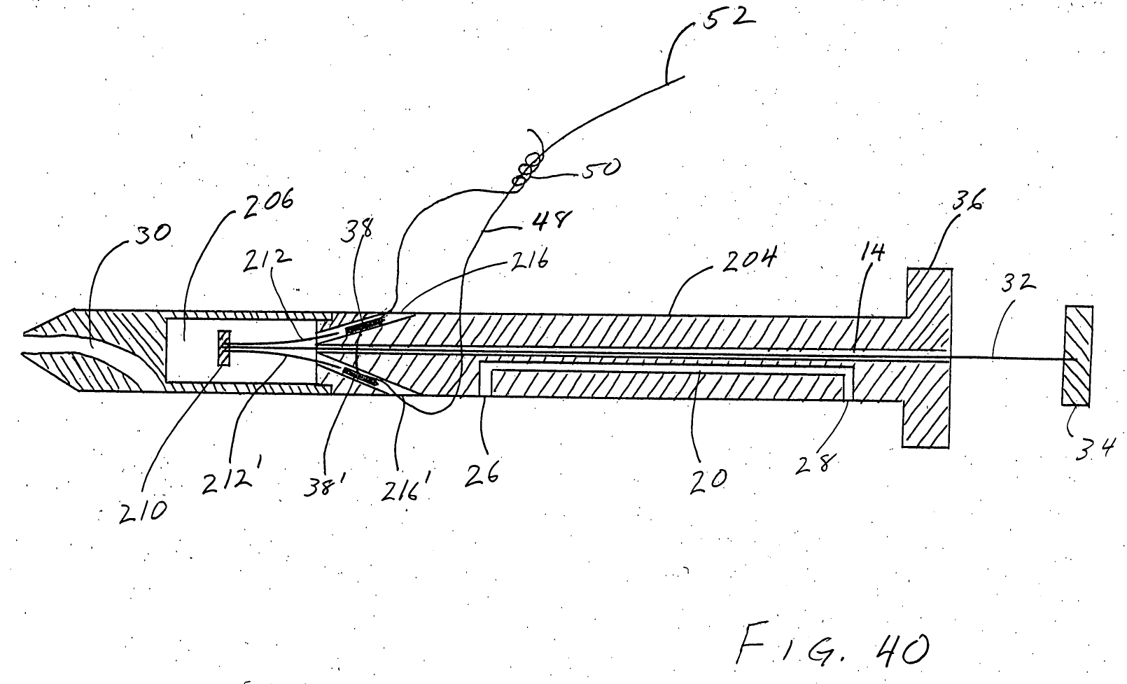

As those of skill in the art will readily appreciate, needles (similar to those shown in the embodiment of FIG. 44) could be used to puncture holes through the internal structure when employing each of the embodiments of the instant invention. In particular, needles could be used to form the anchor recesses in the embodiment of FIG. 1 and a second push rod, similar to the second pull rod of the FIG. 44 embodiment, could be used to operate those needles, thereby using the needles to puncture the wall of the structure before forcing the anchors out of the needles. Similar modifications will be apparent to those of skill in this art with respect to the embodiments of FIGS. 15, 21, 26, 29, 31, 34 and 38.

As those of skill in this art will recognize, there are many variations of the instant invention beyond those described above. A number of the features of one or more embodiments may readily be adapted for use with other embodiments, all of which persons of skill in the art will recognize are within the scope of the claims of this invention. It should be understood that such modifications, variations and related embodiments are all within the broad scope of the instant invention and that this invention is not intended to be limited to the embodiments described herein. Rather, the instant invention is to be limited only by the claims which are set forth below. It should also be understood that, although the above description has been directed to sealing puncture wounds in blood vessels, suturing leaflets of the mitral valve, and securing prostheses, like heart valves and rings in place, the instant invention has applicability to sealing punctures, tears other holes as well as other defects in many other internal structures and organs, as well as suturing a wide variety of other internal structures and organs for othr purposes as well as attaching a wide variety of other prosthetic and repair devices.

What is claimed is:

1. A device for remotely suturing an internal structure of a body comprising an insertion device having a proximal end and a distal end, one or more anchors slidably held in recesses in said device, each of said anchor recesses being associated with an anchor ejection opening, at least one suture connected to said anchors, said at least one suture being sufficiently long as to reach from said internal structure to the outside of said body, and means for ejecting said anchors from said recesses through said anchor ejection openings, said anchor ejecting means being operable from outside said body, said anchor ejection means being adapted to puncture said internal structure and means to permit securing said anchors in place against said internal structure when the operator pulls said at least one of said sutures.

2. The device of claim 1 wherein said anchor rejection recesses are comprised of hollow needles and wherein said hollow needles also comprise said means for puncturing said internal structure.

3. The device of claim 1 further comprising a slide mechanism on said suture to permit said operator to pull the same tight.

4. The device of claim 3 further comprising means for locking said suture in position after it has been pulled tight.

5. The device of claim 3 wherein said slide mechanism is comprised of a slip knot.

6. The device of claim 3 wherein said slide mechanism is comprised of a crimp ferrule.

7. The device of claim 1 wherein said device is provided with a guidewire lumen adapted to slide over a guidewire.

8. The device of claim 1 wherein said ejecting means are comprised of one movement rod associated with each anchor, said device further comprising pushrod retaining means, said movement rods being slidably retained in said movement rod retaining means and further comprising operating means for enabling an operator, from outside said body, to cause said movement rods to move in said retaining means.

9. The device of claim 8 wherein each of said movement rod retaining means are comprised of a lumen in said device, each of said lumens communicating with one of said recesses.

10. The device of claim 1 wherein said anchors are provided with means for preventing retraction thereof after said anchors have passed through said structure.

11. The device of claim 10 wherein said retraction prevention means are comprised of barbs on said anchors.

12. The device of claim 11 wherein said barbs protrude from the peripheries of said anchors.

13. The device of claim 1 further comprising locator means for enabling the operator to determine, from outside said body, when said device is properly located with respect to said structure.

14. The device of claim 13 wherein said locator means comprises an indicator lumen having distal and proximal openings, said distal opening of said indicator lumen located distally of said anchor ejection openings and said proximal opening of said indicator lumen located outside said body whereby the operator can see fluid emerging therefrom when said distal end is inside the structure.

15. The device of claim 13 wherein said locator means comprises an indicator lumen having distal and proximal openings, said distal opening of said indicator lumen located proximally of said anchor ejection openings and said proximal opening of said indicator lumen located outside said body whereby the operator can see fluid emerging therefrom when said distal end is inside the structure.

16. The device of claim 13 wherein said locator means comprises a barb operable from outside said body to move said locating barb from a retracted position to a deployed position, wherein when in its retracted position said barb is housed within said device and when in its deployed position said locator barb protrudes and splays outwardly from the body of said insertion device, whereby said locator barb, when in its deployed position, engages the wall of said internal organ when said device is properly located in said body whereby the operator can feel said wall engagement of said locator barb, thereby knowing said insertion device is properly located.

17. The device of claim 2 further comprising needle movement means for causing said needles to puncture said internal structure and anchor movement means for causing said anchors to exit said needles after said needles have punctured said structure.

18. The device of claim 17 wherein said needle movement means are comprised of one movement rod associated with each needle, said device further comprising needle movement rod retaining means, wherein said needle movement rods are slidably retained in said needle rod retaining means and further comprising needle operating means for enabling an operator, from outside said body, to cause said needle movement rods to move in said needle movement rod retaining means.

19. The device of claim 18 wherein said anchor movement means are comprised of one anchor movement rod associated with each of said anchors, said device further comprising anchor movement rod retaining means, wherein said anchor movement rods are slidably retained in said anchor movement rod retaining means, and further comprising anchor operating means for enabling an operator, from outside said body, to cause said anchor movement rods to move in said anchor movement rod retaining means.

20. The device of claim 1 having two or more anchors wherein said anchor ejection openings are spaced approximately equidistant around the periphery of said device.

21. The device of claim 17 further comprising locator means for enabling the operator to determine, from outside said body, when said device is properly located with respect to said structure.

22. The device of claim 1 having two anchors.

23. The device of claim 22 wherein said anchor ejection openings are spaced approximately 180° apart around the periphery of said device.

24. The device of claim 1 having three anchors.

25. The device of claim 24 wherein said anchor ejecting openings are spaced approximately 120° apart around the periphery of said device.

26. The device of claim 1 having four anchors.

27. The device of claim 26 wherein said anchor ejecting openings are space approximately 90° apart around the periphery of said device.

28. The device of claim 1 further comprising an expansion section on which said anchor recesses are housed, said expansion section being expandable from a collapsed position to an expanded position.

29. The device of claim 28 wherein said expansion section is comprised of an inflatable balloon.

30. The device of claim 28 wherein said expansion section is self expanding from said collapsed position to said expanded position.

31. The device of claim 28 wherein said expansion section is comprised of a scaffold having multiple struts.

32. The device of claim 8 wherein said operating means causes all of said pushrods to move substantially simultaneously.

33. The device of claim 8 wherein said operating means enables said pushrods to be moved independently.

34. The device of claim 1 comprising at least two anchors wherein said device is rotatable from a first position to a second position while inside said body, said device adapted to have one of said anchors ejected from an anchor ejection opening when said device is in said first position and to have another of said anchors ejected from an ejection opening when said device is in said second position.

35. The device of claim 34 wherein there is only one anchor ejection opening and wherein said anchors are stored in said device for seriatim discharge through said ejection opening, further comprising means for ejecting one anchor when said device is in said first position, means for readying a second anchor for ejection after said first anchor has been ejected.

36. The device of claim 34 comprising two or more suture anchors slidably held in recesses in said device, at least one of said anchor recesses communicating with an anchor ejection opening, a suture connecting said anchors, means for ejecting said suture anchors from said recesses through at least one of said anchor ejection openings, said suture anchors being adapted to puncture said internal structure, a stabilizer resident in a lumen of said device, said stabilizer comprising an operating handle adapted to move said stabilizer from a retracted position to a deployed position and from said deployed position to said retracted position and further comprising a stabilizing anchor adapted, when said device is in said first position, to stabilize a first section of said internal structure when said stabilizer is moved from said retracted position to said deployed position and to hold said first section of said structure relatively stable while said stabilizer is in said deployed position, said ejecting means adapted to eject at least one of said suture anchors through one of said ejection openings and into and through said first section of said internal structure while said stabilizing anchor is stabilizing said first section, said stabilizing anchor adapted to release said first section of said internal structure when said stabilizer is moved to said retracted position, said stabilizing anchor adapted to stabilize a second section of said internal structure when said device has been rotated to said second position and said stabilizer has been moved to said deployed position, means for ejecting at least a second suture anchor through an ejection opening and causing said second suture anchor to puncture said second section, said stabilizing anchor adapted to release said second section of said internal structure when said stabilizer is moved to said retracted position, a slide mechanism on said suture to permit pulling said anchors toward one another when tension is applied to one or both ends of said suture.

37. The device of claim 36 wherein said stabilizing anchor is adapted to stabilize said internal structure by piercing the same and holding it impaled on said stabilizing anchor.

38. The device of claim 36 wherein said stabilizing anchor is adapted to stabilize said internal structure by having suction applied through said stabilizing anchor to said structure to hold same against said stabilizing anchor.

39. The device of claim 36 wherein said slide mechanism is comprised of a slip knot on said suture.

40. The device of claim 36 wherein said slide mechanism is comprised of a crimp ferrule.

41. The device of claim 1 wherein said anchors are slidably mounted on a common suture.

42. The device of claim 1 wherein each of said anchors is mounted on its own suture.

43. The device of claim 1 further comprising a stabilizing anchor operable from outside said body to move said stabilizing anchor from a retracted position to a deployed position.

44. The device of claim 43 wherein when said stabilizing anchor is in its retracted position, it is housed within a lumen of said device.

45. The device of claim 43 wherein said stabilizing anchor is operable from outside said body to move said stabilizing anchor from said deployed position to said retracted position.

46. The device of claim 43 further comprising an lumen communicating from outside said body through said stabilizer and said stabilizing anchor and opening at the end of said stabilizing anchor wherein said lumen can be used to apply suction to said structure when the same is in contact with said stabilizing anchor whereby said structure is stabilized.

47. The device of claim 43 further comprising a guidewire lumen.

48. The device of claim 1 wherein said device is designed to close an opening in said internal structure.

49. The device of claim 48 wherein said device is comprised of at least two anchors, wherein all of said anchors are mounted on a common suture and wherein when the operator pulls said suture tight said anchors are pulled toward one another, thereby closing said opening.

50. The device of claim 1 further comprising a prosthetic device mounted on said at least one suture, wherein when said operator pulls on said suture, said prosthetic device is secured in place on said internal structure.

51. The device of claim 50 wherein said device is comprised of at least two anchors, wherein all of said anchors are mounted on a common suture and wherein when said operator pulls said suture tight said anchors are secured to said structure, thereby securing said prosthesis in place.

52. The device of claim 50 wherein said device is comprised of at least two anchors, wherein each of said anchors is mounted on its own suture and wherein when said operator pulls said sutures tight said anchors are secured to said structure, thereby securing said prosthesis in place.

53. The device of claim 1 wherein said device is designed to reinforce a weakened portion of said internal structure by suturing a sheet material to said internal structure over said weakened portion, thereby to reinforce said portion.

54. The device of claim 53 wherein said device is comprised of at least two anchors, wherein all of said anchors are mounted on a common suture and wherein when said operator pulls said suture tight said anchors are secured to said structure, thereby securing said sheet material in place.

55. The device of claim 53 wherein said device is comprised of at least two anchors, wherein each said anchors is mounted on its own suture and wherein when said operator pulls said sutures tight said anchors are secured to said structure, thereby securing said sheet material in place.

56. The device of claim 53 further comprising locator means for enabling the operator to determine, from outside said body, when said device is properly located with respect to said structure.

57. The device of claim 56 wherein said locator means comprises an indicator lumen having distal and proximal openings, said distal opening of said indicator lumen located distally of said anchor ejection openings and said proximal opening of said indicator lumen located outside said body whereby the operator can see fluid emerging therefrom when said distal end is inside the structure.

58. The device of claim 56 wherein said locator means comprises an indicator lumen having distal and proximal openings, said distal opening of said indicator lumen located proximally of said anchor ejection openings and said proximal opening of said indicator lumen located outside said body whereby the operator can see fluid emerging therefrom when said distal end is inside the structure.

59. The device of claim 56 wherein said locator means comprises wherein said locator means comprises a barb operable from outside said body to move said locator barb from a retracted position to a deployed position, wherein when in its retracted position said barb is housed within said device and when in its deployed position said locator barb protrudes and splays outwardly from the body of said insertion device, whereby said locator barb, when in its deployed position, engages the wall of said internal organ when said device is properly located in said body whereby the operator can feel said wall engagement of said locator barb, thereby knowing said insertion device is properly located.

60. A method for remotely suturing, from outside a body, an internal structure in said body, comprising the steps of:
a. from outside of said body, inserting into said body a device comprising at least one anchor, at least one suture, at least one anchor recess and at least one anchor ejection opening,
b. positioning said device so that at least one of said anchor ejection openings is adjacent said structure,
c. puncturing a hole in said anatomical structure;
d. causing an anchor to exit at least one of said anchor ejection openings and pass through one of said puncture holes in said anatomical structure, each of said anchors being mounted on a suture,
e. withdrawing said device from said body, leaving at least one of said anchors mounted on said at least one suture adjacent said structure, and
f. from outside of the said body, tightening said at least one suture to secure said anchors in place.

61. The method of claim 60 wherein said anchors are slidably mounted on a common suture having two ends and wherein the ends of said suture are slidably connected outside of said structure, applying tension to one end of said at least one suture to secure said anchors in place.

62. The method of claim 60 wherein there are at least two anchors, further comprising the steps of positioning said ejection openings adjacent a wound in said structure, causing non-adjacent puncture holes to be made in said structure, tightening up on said suture to draw said anchors toward one another, thereby repairing said wound.

63. The method of claim 62 wherein there are three anchors, further comprising the step of causing said anchors to pass through their respective puncture holes in said structure spaced approximately 120° from one another.

64. The method of claim 62 wherein there are four anchors further comprising the step of causing said anchors to pass through their respective puncture holes in said structure spaced approximately 90° from one another.

65. The method of claim 62 wherein said anchors are caused to exit said anchor ejection openings approximately simultaneously.

66. The method of claim 62 wherein said anchors are caused to exit said ejection openings independently.

67. The method of claim 60 further comprising the step of inserting said device into said body until locator means indicate that said device is properly located adjacent said structure.

68. The method of claim 62 further comprising the step of exerting, from outside said body, distal force on at least one movement member to cause said anchors to exit said anchor ejection openings.

69. The method of claim 68 wherein said force is applied to said movement members by moving a common operating handle to which all movement members are attached.

70. The method of claim 60 further comprising the steps of, before ejecting said anchors, enlarging an expansion section of said device and centering said expansion section in an opening in said structure.

71. The method of claim 70 wherein said expansion section is comprised of a balloon and further comprising the step of expanding said section by inflating said balloon.

72. The method of claim 70 wherein said expansion section is comprised of a multi-strut scaffold and further comprising the steps of applying force to collapse said section before inserting said device into said body and expanding said scaffold when said device is properly located in said body.

73. The method of claim 72 wherein said scaffold is self-expanding and wherein the step of expanding said scaffold is accomplished by releasing said force.

74. The device of claim 1 wherein said device is designed to have said anchor ejection openings located on the outside of said structure when said anchors are ejected.

75. The device of claim 74 wherein said device is adapted to have said anchors puncture said structure from the outside thereof to the inside thereof and wherein said anchors remain inside said structure when said operator pulls said at least one said suture.

76. The device of claim 1 wherein said device is designed to have said anchor ejection openings pass into said internal structure before said anchors are ejected therefrom.

77. The device of claim 76 wherein said anchor ejection means are adapted to puncture said structure from the inside thereof to the outside thereof and wherein said anchors remain outside said structure when said operator pulls said at least one said suture.

78. The method of claim 60 wherein said structure has a wall, wherein said positioning step places said at least one ejection opening outside of said wall of said structure, wherein said puncture step is accomplished by having said at least one said anchor, when it exits said at least one ejection opening, make its own hole in said wall of said structure from the outside thereof to the inside thereof, and wherein said withdrawing step leaves said at least one anchor inside said wall of said structure.

79. The method of claim 60 wherein said structure has a wall, wherein said positioning step places said at least one ejection opening inside of said wall of said structure, wherein when said at least one said anchor exits said at least one ejection opening it makes its own hole in said wall of said structure from the inside thereof to the outside thereof, and wherein said withdrawing step leaves said at least one anchor outside said wall of said structure.

80. The method of claim 60 wherein said structure has a wall defining the perimeter thereof, wherein said positioning step places said at least one said ejection opening outside of said wall of said structure, wherein said puncturing step is accomplished by forcing at least one needle through said wall from the outside thereof to the inside thereof, thereby making a puncture hole for at least one of said anchors, and wherein said withdrawing step leaves said at lease one anchor inside said structure.

81. The method of claim 60 wherein said structure has a wall defining the perimeter thereof, wherein said positioning step places said at least one said ejection opening inside of said wall of said structure, wherein said puncturing step is accomplished by forcing at least one needle through said wall from the inside thereof to the outside thereof, thereby making a puncture hole for at least one of said anchors, and wherein said withdrawing step leaves said at lease one anchor outside said structure.
